(19) United States
(12) Patent Application Publication
(10) Pub. No.: US 2025/0041247 A1
(43) Pub. Date: Feb. 6, 2025

(54) SMALL MOLECULE INHIBITORS OF REPEAT ASSOCIATED NON-AUG (RAN) TRANSLATION AND COMBINATION THERAPIES

(71) Applicant: University of Florida Research Foundation, Incorporated, Gainesville, FL (US)

(72) Inventors: Laura Ranum, Gainesville, FL (US); Tao Zu, Gainesville, FL (US)

(73) Assignee: University of Florida Research Foundation, Incorporated, Gainesville, FL (US)

(21) Appl. No.: 18/715,436

(22) PCT Filed: Dec. 1, 2022
(86) PCT No.: PCT/US2022/051530

§ 371 Date: May 31, 2024

Related U.S. Application Data
(60) Provisional application No. 63/284,762, filed on Dec. 1, 2021.

(51) Int. Cl. A61K 31/155; A61K 31/352; A61K 31/495; A61P 25/28

(52) U.S. Cl. CPC A61K 31/155; A61K 31/352; A61K 31/495; A61P 25/28

(57) ABSTRACT

The present disclosure provides methods, uses, kits, and compositions comprising a compound of Formula (I), and either a second compound of Formula (I) or a compound of Formula (II). In one aspect, the methods are for treating a neurological disease associated with repeat expansions and/or RAN protein accumulation, reducing the level of one or more repeat associated non-AUG (RAN) proteins, and reducing the accumulation of RAN proteins in a subject and/or biological sample. Exemplary diseases associated with repeat expansions include, but are not limited to, amyotrophic lateral sclerosis (ALS), frontotemporal dementia, myotonic dystrophy type 1, myotonic dystrophy type 2, spinocerebellar ataxia, Alzheimer's disease, Huntington's disease, Fragile X Tremor Ataxia Syndrome, and Fragile XE syndrome.

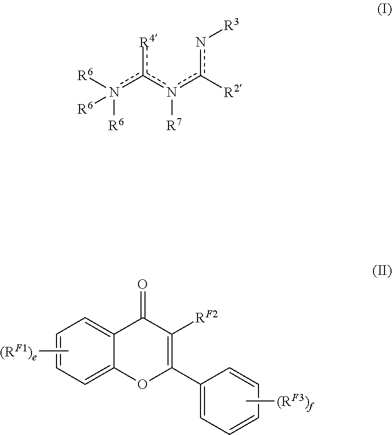

### RELATED APPLICATIONS

This application claims priority under 35 U.S.C. § 119(e) to U.S. Provisional Application, U.S. Ser. No. 63/284,762, filed Dec. 1, 2021, which is incorporated herein by reference.

### GOVERNMENT SUPPORT

This invention was made with government support under grant number W81XWH-19-1-0654, awarded by the U.S. Army Medical Research Acquisition Activity. The government has certain rights in the invention.

### BACKGROUND OF THE INVENTION

Mutations of certain repeat expansions (e.g., CAGG, CCTG, GGGGCC, GGCCCC, CAG, and CTG) are associated with a number of different neurological diseases (e.g., C9ORFf72 amyotrophic lateral sclerosis (ALS); C9ORF72 frontotemporal dementia; Alzheimer's disease; myotonic dystrophy type 1 (DM1); myotonic dystrophy type 2 (DM2); spinocerebellar ataxia types 1, 2, 3, 6, 7, 8, 10, 12, 17, 31, and 36; spinal bulbar muscular atrophy; dentatorubral-pallidoluysian atrophy (DRPLA); Huntington's disease (HD); Fragile X Tremor Ataxia Syndrome (FXTAS)); Fuch's endothelial corneal dystrophy (FECD); Huntington's disease-like 2 syndrome (HDL2); Fragile X syndrome (FXS); disorders related to 7p11.2 folate-sensitive fragile site FRA7A; disorders related to folate-sensitive fragile site 2q11 FRA2A; and Fragile XE syndrome (FRAXE)). In a growing number of these diseases including, but not limited to C9ORF72 ALS, C9ORF72 FTD, FXTAS, HD, SCA8, DM1, and DM2, expansion mutations have been shown to undergo a novel type of protein translation that occurs in multiple reading frames and does not require a canonical AUG initiation codon (Zu et al. 2011; Ash et al. 2013; Mori et al. 2013; Todd et al. 2013; Zu et al. 2013; Banez-Coronel et al. 2015; Cleary and Ranum 2017; Zu et al. 2017). There is growing evidence that RAN proteins are toxic and contribute to a growing number of diseases (Cleary and Ranum 2017). Therefore, it is important to develop therapeutic strategies that reduce the level of RAN proteins to treat neurological diseases caused by repeat expansion mutations. Some strategies include those described in: PCT Publication WO2019/067587, PCT Publication WO2021/007110, and PCT Publication WO2021/231887, each of which is incorporated herein by reference in its entirety.

### SUMMARY OF THE INVENTION

Described herein are compositions, uses, kits, and methods for the treatment and prevention of neurological diseases associated with repeat associated non-AUG (RAN) proteins. The compositions, uses, kits, and methods comprise a combination of compounds (e.g., analogs of metformin and analogs of quercetin). Mutations of repeat expansions (e.g., CAGG, CCTG, GGGGCC, GGCCCC, CAG, and CTG) are associated with a number of different neurological diseases (e.g., C9ORFf72 amyotrophic lateral sclerosis (ALS); C9ORF72 frontotemporal dementia (FTD); Alzheimer's disease; myotonic dystrophy type 1 (DM1); myotonic dystrophy type 2 (DM2); spinocerebellar ataxia types 1, 2, 3, 6, 7, 8, 10, 12, 17, 31, and 36; spinal bulbar muscular atrophy; dentatorubral-pallidoluysian atrophy (DRPLA); Huntington's disease (HD); Fuch's endothelial corneal dystrophy (FECD); Fragile X Tremor Ataxia Syndrome (FXTAS); Huntington's disease-like 2 syndrome (HDL2); Fragile X syndrome (FXS); disorders related to 7p11.2 folate-sensitive fragile site FRA7A; disorders related to folate-sensitive fragile site 2q11 FRA2A; and Fragile XE syndrome (FRAXE)). In particular, four RAN translation proteins-polyalanine, polyserine, polyleucine, and polycysteine (poly(Ala), poly(Ser), poly(Leu), and poly(Cys), respectively)-accumulate in the brain, tissue, fluids (e.g., blood, cerebrospinal fluid (CSF)), and central nervous systems of subjects having Huntington's disease (HD). In C9ORF72 ALS or C9ORF72 FTD, RAN proteins with dipeptide repeats (e.g., polyGlyPro (GP), polyGlyAla (GA), polyGlyArg (GR), polyProAla (PA)) have been shown to accumulate in patients' brains, blood, and other tissues. Similarly, homopolymeric and tetrapeptide RAN proteins have been found in patients with Fragile X Tremor Ataxia Syndrome (FXTAS), myotonic dystrophy type 1 (DM1), and myotonic dystrophy type 2 (DM2). Based on results from Zu (Zu et al., PNAS 2011), RAN proteins are also predicted to accumulate in patients with diseases caused by CAG/CTG repeat expansions including, but not limited to, spinocerebellar ataxia types 1, 2, 3, 6, 7, 8, 10, 12, 17, 31, and 36; spinal bulbar muscular atrophy (SBMA); dentatorubral-pallidoluysian atrophy (DRPLA); and Fuch's corneal endothelial dystrophy. RAN proteins can be detected in a biological sample (e.g., blood, serum, tissue, or cerebrospinal fluid (CSF)) from a subject having or at risk of developing various diseases (e.g., Huntington's Disease; C9ORF72 ALS; C9ORF72 FTD; DM1; DM2; FXTAS; SCA8; Huntington's disease-like 2 syndrome (HDL2); Fragile X syndrome (FXS); disorders related to 7p11.2 folate-sensitive fragile site FRA7A; disorders related to folate-sensitive fragile site 2q11 FRA2A; Fragile XE syndrome (FRAXE); or other diseases caused by microsatellite repeat expansion mutations).

In one aspect, provided herein are methods of treating or preventing a neurological disease associated with repeat expansions in a subject in need thereof, the method comprising administering to the subject a combination of two compounds (e.g., two separate analogs of metformin or analogs of metformin and analogs of quercetin) or quercetin alone.

In one aspect, provided herein are methods of treating or preventing a neurological disease associated with repeat expansions in a subject in need thereof, the method comprising administering to the subject metformin, or analog thereof (i.e., a compound of Formula (I)), and a second analog of metformin (i.e., a compound of Formula (I)). In one aspect, provided herein are methods of treating or preventing a neurological disease associated with repeat expansions in a subject in need thereof, the method comprising administering to the subject:
(a) a compound of Formula (I):

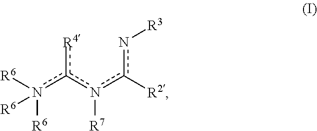

or a pharmaceutically acceptable salt, co-crystal, tautomer, stereoisomer, solvate, hydrate, polymorph, isotopically enriched derivative, or prodrug thereof, wherein [symbol] , R^{2′}, R³, R^{4′}, R⁶, and R⁷ are defined herein; and
(b) a second compound of Formula (I), or a pharmaceutically acceptable salt, co-crystal, tautomer, stereoisomer, solvate, hydrate, polymorph, isotopically enriched derivative, or prodrug thereof.

In one aspect, provided herein are methods of treating or preventing a neurological disease associated with repeat expansions in a subject in need thereof, the method comprising administering to the subject metformin, or analog thereof (i.e., a compound of Formula (I)), and quercetin, or analog thereof (i.e., a compound of Formula (II)). In another aspect, provided herein are methods of treating or preventing a neurological disease associated with repeat expansions in a subject in need thereof, the method comprising administering to the subject:
(a) a compound of Formula (I):

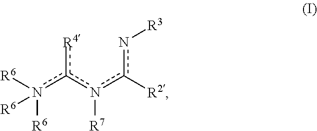

or a pharmaceutically acceptable salt, co-crystal, tautomer, stereoisomer, solvate, hydrate, polymorph, isotopically enriched derivative, or prodrug thereof, wherein [symbol] , R^{2′}, R³, R^{4′}, R⁶, and R⁷ are defined herein; and
(b) a compound of Formula (II):

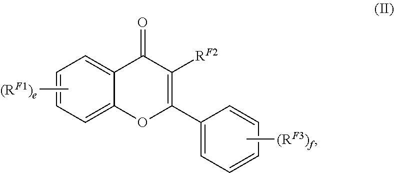

or a pharmaceutically acceptable salt, co-crystal, tautomer, stereoisomer, solvate, hydrate, polymorph, isotopically enriched derivative, or prodrug thereof, wherein R^{F1}, R^{F2}, R^{F3}, e, and f are as defined herein.

In one aspect, provided herein are methods of treating or preventing a neurological disease associated with repeat expansions in a subject in need thereof, the method comprising administering to quercetin, or analog thereof (i.e., a compound of Formula (II)). In another aspect, provided herein are methods of treating or preventing a neurological disease associated with repeat expansions in a subject in need thereof, the method comprising administering to the subject a compound of Formula (II):

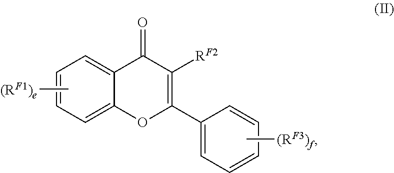

or a pharmaceutically acceptable salt, co-crystal, tautomer, stereoisomer, solvate, hydrate, polymorph, isotopically enriched derivative, or prodrug thereof, wherein R^{F1}, R^{F2}, R^{F3}, e, and f are as defined herein.

The disclosure also provides pharmaceutical compositions comprising two or more compounds.

In some aspects, the disclosure provides a composition comprising metformin, or analog thereof (i.e., a compound of Formula (I)), and a second analog of metformin (i.e., a compound of Formula (I)). In one aspect, provided herein are pharmaceutical compositions comprising: (a) a compound of Formula I):

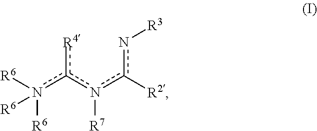

or a pharmaceutically acceptable salt, co-crystal, tautomer, stereoisomer, solvate, hydrate, polymorph, isotopically enriched derivative, or prodrug thereof, wherein [symbol] , R², R³, R⁴, R⁶, and R⁷ are defined herein; and (b) a second compound of Formula (I), or a pharmaceutically acceptable salt, co-crystal, tautomer, stereoisomer, solvate, hydrate, polymorph, isotopically enriched derivative, or prodrug thereof.

In some aspects, the disclosure provides a composition comprising metformin, or analog thereof (i.e., a compound of Formula (I)), and quercetin, or analog thereof (i.e., a compound of Formula (II)). In one aspect, provided herein are pharmaceutical compositions comprising: (a) a compound of Formula (I):

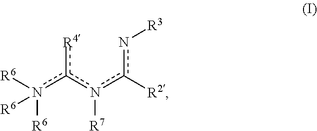

or a pharmaceutically acceptable salt, co-crystal, tautomer, stereoisomer, solvate, hydrate, polymorph, isotopically enriched derivative, or prodrug thereof, wherein [symbol] , R^{2′}, R³, R^{4′}, R⁶, and R⁷ are defined herein; and (b) a compound of Formula (II):

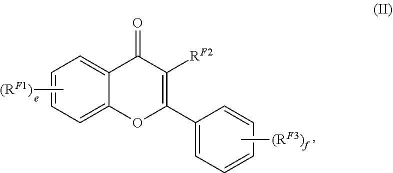

or a pharmaceutically acceptable salt, co-crystal, tautomer, stereoisomer, solvate, hydrate, polymorph, isotopically enriched derivative, or prodrug thereof, wherein R^{F1}, R^{F2}, R^{F3}, e, and f are as defined herein.

In some embodiments, the compound of Formula (I) is:

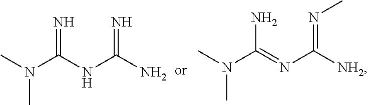

or a pharmaceutically acceptable salt, co-crystal, tautomer, stereoisomer, solvate, hydrate, polymorph, isotopically enriched derivative, or prodrug thereof. In some embodiments, the second compound of Formula (I) is:

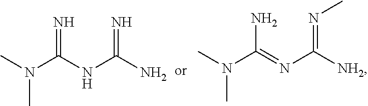

or a pharmaceutically acceptable salt, co-crystal, tautomer, stereoisomer, solvate, hydrate, polymorph, isotopically enriched derivative, or prodrug thereof. In certain embodiments, the compound of Formula (II) is:

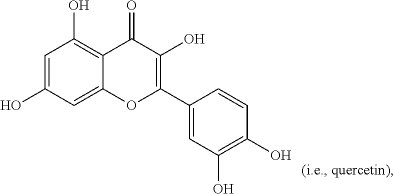 [SMILES: O=c1c(O)c(-c2ccc(O)c(O)c2)oc2cc(O)cc(O)c12]

or a pharmaceutically acceptable salt, co-crystal, tautomer, stereoisomer, solvate, hydrate, polymorph, isotopically enriched derivative, or prodrug thereof.

In some embodiments, the methods and compositions are used for treating and/or preventing Alzheimer's disease, amyotrophic lateral sclerosis (ALS), frontotemporal dementia (FTD), spinocerebellar ataxia, myotonic dystrophy type 1, myotonic dystrophy type 2, Fuch's corneal endothelial dystrophy, spinal bulbar muscular atrophy, dentatorubral-pallidoluysian atrophy, Huntington's disease, Fragile X Tremor Ataxia Syndrome (FXTAS), Huntington's disease-like 2 syndrome (HDL2), Fragile X syndrome (FXS), disorders related to 7p11.2 folate-sensitive fragile site FRA7A, disorders related to folate-sensitive fragile site 2q11 FRA2A, or Fragile XE syndrome (FRAXE).

Another aspect of the disclosure relates to methods of reducing the accumulation of repeat associated non-AUG protein (RAN) in a subject or biological sample (e.g., tissue, organ, or cell), the method comprising administering to the subject, or contacting the biological sample (e.g., tissue, organ, or cells) with an effective amount of a compound of Formula (I) and either a second compound of Formula (I) or a compound of Formula (II), or a pharmaceutically acceptable salt, solvate, hydrate, tautomer, stereoisomer, derivative, or prodrug thereof, or a pharmaceutical composition thereof.

The present disclosure also provides uses of a compound of Formula (I) and either a second compound of Formula (I) or a compound of Formula (II), or a pharmaceutically acceptable salt, solvate, hydrate, tautomer, stereoisomer, derivative, or prodrug thereof, or a pharmaceutical composition thereof, to treat and/or prevent a neurological disease associated with repeat expansions in a subject in need thereof.

Another aspect of the present disclosure relates to kits comprising a container with a compound of Formula (I) and either a second compound of Formula (I) or a compound of Formula (II), or a pharmaceutically acceptable salt, solvate, hydrate, tautomer, stereoisomer, derivative, or prodrug thereof, or a pharmaceutical composition thereof, as described herein.

The details of one or more embodiments of the disclosure are set forth herein. Other features, objects, and advantages of the disclosure will be apparent from the Detailed Description, Examples, Figures, and Claims.

### BRIEF DESCRIPTION OF THE DRAWINGS

FIG. 1 depicts the chemical structures of metformin, Compound 3, which is a compound of Formula (I), and quercetin.

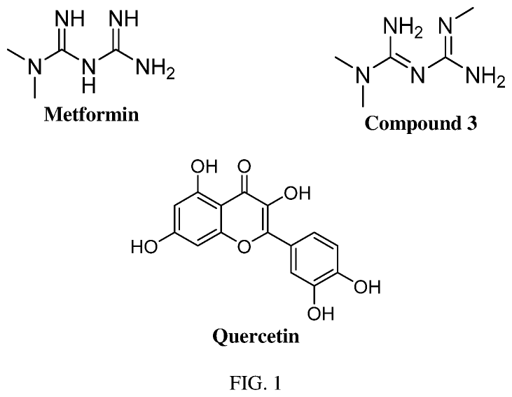

FIG. 2 shows that the combined use of Compound 3 and metformin synergistically decreases p-PKR and poly-Ala RAN proteins, as well as RAN and ATG-initiated poly-Gln protein levels. Protein blots of HEK293T cells transiently transfected with CAG expansion constructs were treated with different concentrations of Compound 3, with or without 5 mM metformin.

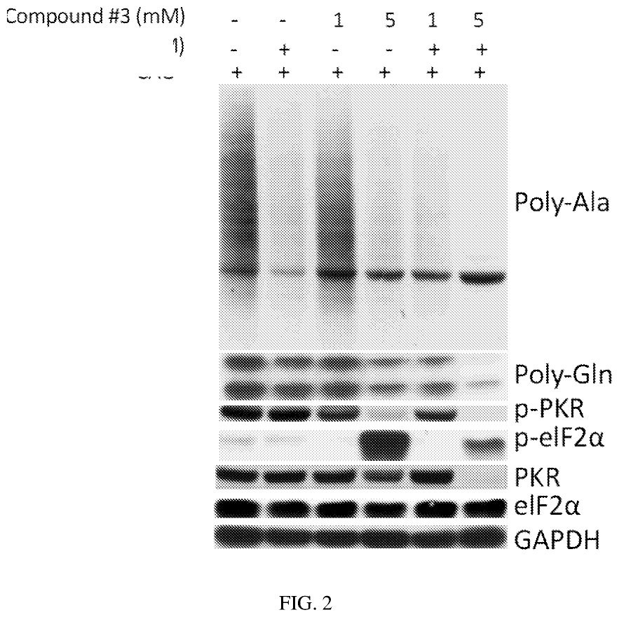

FIG. 3 shows that the combined use of Compound 3 and metformin synergically decreases p-PKR and poly-Ala RAN proteins, as well as RAN polyGln and ATG-initiated poly-Gln. Protein blots of HEK293T cells transiently transfected with CAG expansion constructs were treated with different concentrations of compound 3, with or without metformin.

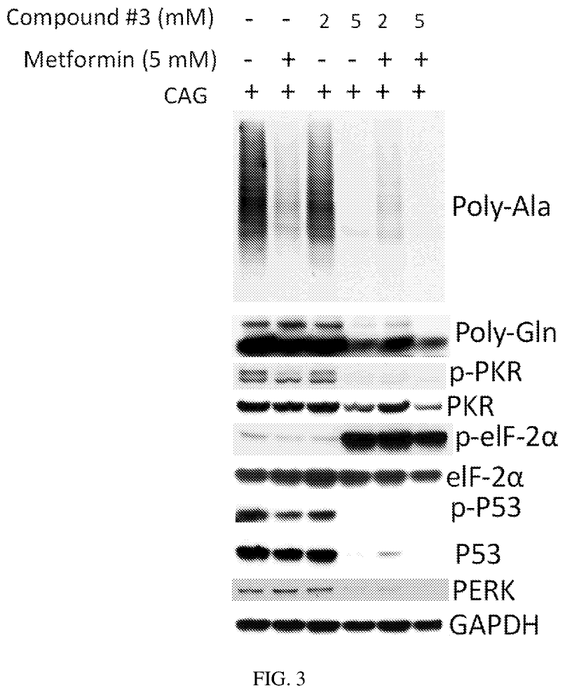

FIG. 4 shows that the combined use of quercetin and metformin synergically decreases poly-Ala RAN proteins, as well as RAN polyGln and ATG-initiated poly-Gln. Protein blots of HEK293T cells transiently transfected with CAG expansion constructs were treated with different concentrations of quercetin, with or without metformin.

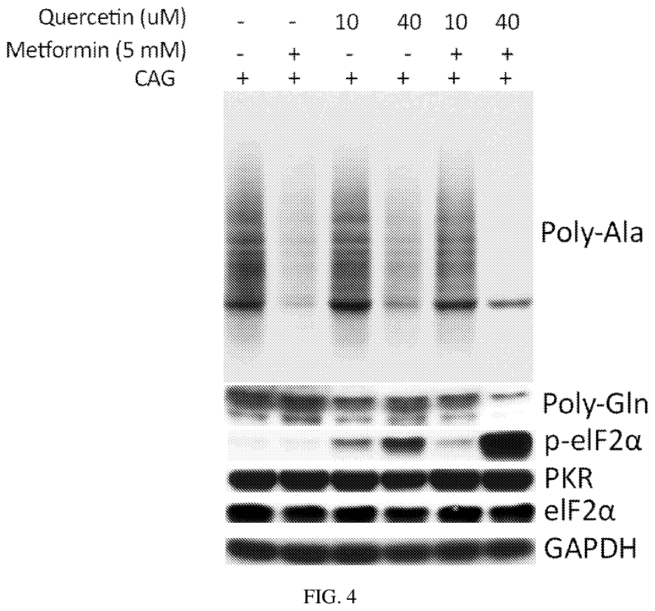

FIG. 5 shows that the combined use of quercetin and metformin synergistically decreases p-PKR, poly-Ala RAN proteins, RAN polyGln and ATG-initiated poly-Gln, p53 and PERK but increases p-eIF2α levels. Protein blots of HEK293T cells transiently transfected with CAG expansion constructs treated with different concentrations of quercetin, with or without metformin.

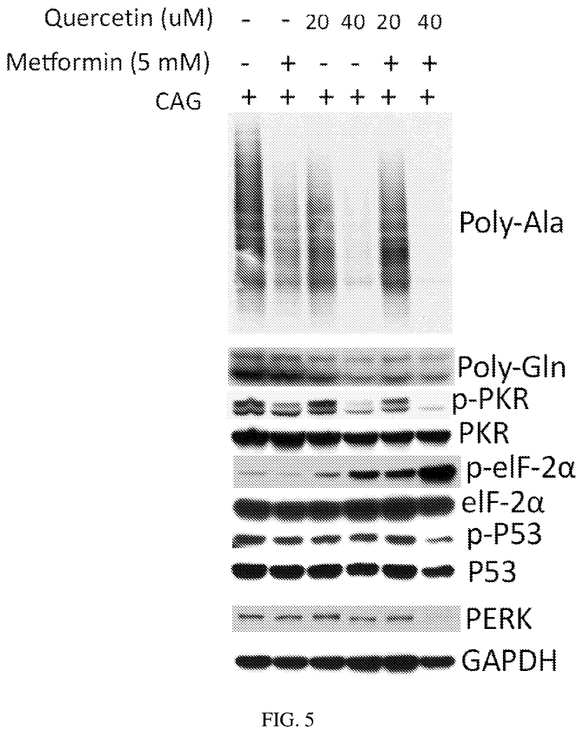

FIG. 6 and FIG. 7 show that the combined use of quercetin and metformin synergistically decreases p-PKR and polyAla RAN proteins in a concentration dependent manner and at the highest quercetin concentrations, p-eIF2α increases. Protein blots of HEK293T cells transiently transfected with CAG expansion constructs treated with different concentrations of quercetin, with or without metformin.

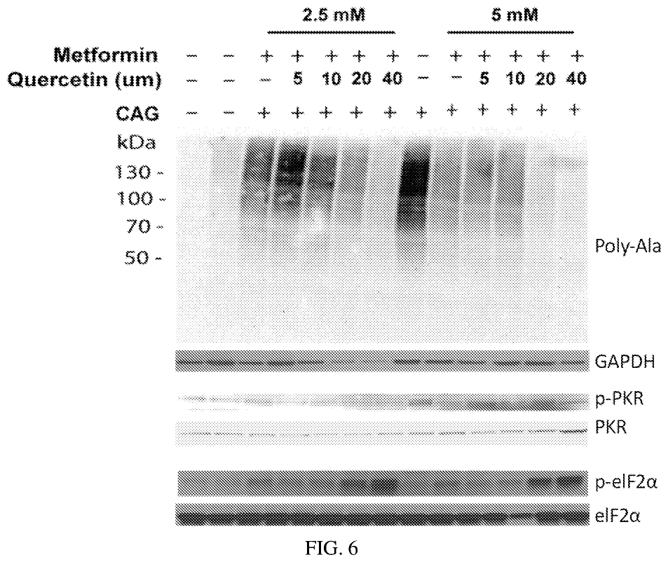

FIG. 8 and FIG. 9 show that the combined treatment of quercetin and metformin results in decreased p-PKR levels, which is greater than that observed with metformin alone. FIG. 8 shows protein blots of HEK293T cells transiently transfected with CAG expansion constructs treated with different concentrations of quercetin, with and without metformin.

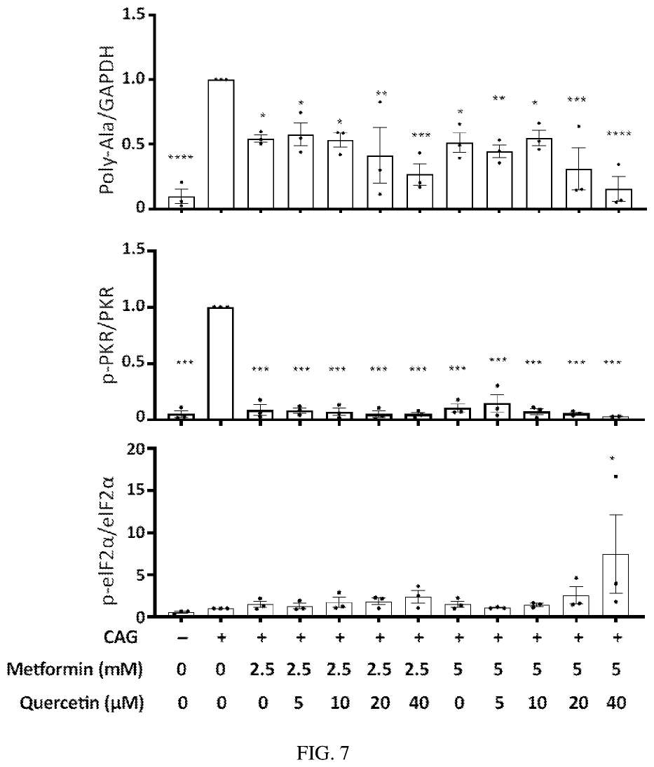

FIG. 9 shows a graphical representation of data obtained from multiple experiments with example results represented in FIG. 8.

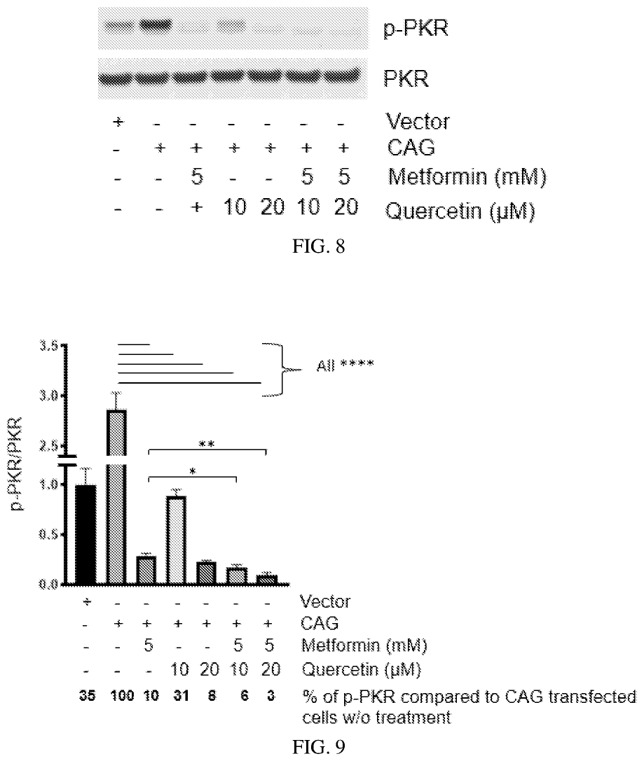

FIG. 10 shows the reduction of polyAla RAN proteins expressed from (CAG)exp with metformin and quercetin treatment. Protein blots of HEK293T cells transiently transfected with CAG expansion constructs after treatment with different concentrations of quercetin, or metformin.

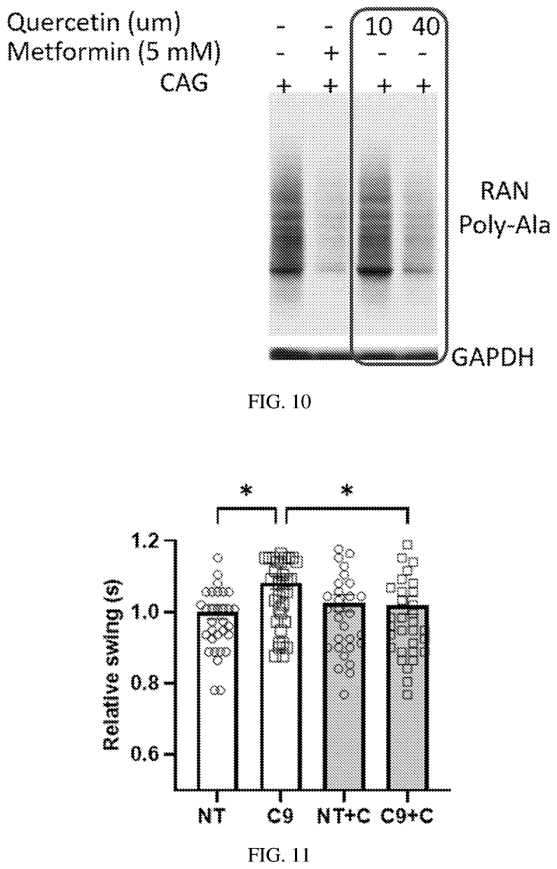

FIGS. 11-13 show gait improvements in C9orf72 ALS mice treated with quercetin, referred to as compound “C” in these figures. All 13 gait abnormalities found between C9orf72 ALS/FTD mice (C9) vs. non-transgenic (NT) mice are corrected by quercetin treatment (see Table 1). FIG. 11 shows the relative swing of non-transgenic mice (NT) and C9orf72 ALS/FTD mice (C9), with and without treatment with quercetin (C). FIG. 12 shows the relative stride length of non-transgenic mice (NT) compared to C9orf72 ALS/FTD mice (C9), with and without treatment with quercetin (C). FIG. 13 shows the relative stride frequency of non-transgenic mice (NT) and C9orf72 ALS/FTD mice (C9), with and without treatment with quercetin (C). Table 1 shows each of the 13 DigiGait abnormalities in the mock treatment group were corrected with quercetin treatment.

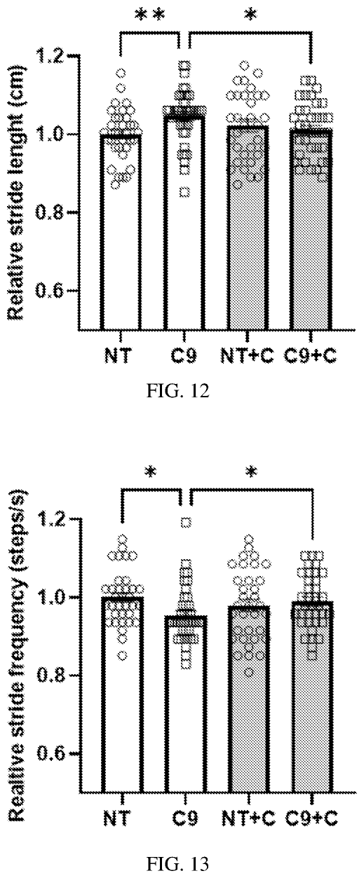

### DEFINITIONS

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The disclosure additionally encompasses compounds as individual isomers substantially free of other isomers, and alternatively, as mixtures of various isomers.

In a formula, the bond [symbol] is a single bond, the dashed line - - - is a single bond or absent, and the bond [symbol] or [symbol] is a single or double bond.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F (i.e., a haloalkyl). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂C₁, and the like.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not limited in any manner by the exemplary substituents described herein.

Exemplary carbon atom substituents include halogen, —CN, —NO₂, —N₃, —SO₂H, —SO₃H, —OH, —OR^{aa}—, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}—, —NR^{bb}CO₂R^{aa}, —NR^{bb}C(═O)N(R^{bb})₂, —C(═NR^{bb})R^{aa}, —C(═NR^{bb})OR^{aa}—, —OC(═NR^{bb})R^{aa}, —OC(═NR^{bb})OR^{aa}—, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 Rad groups; wherein X⁻ is a counterion;
or two geminal hydrogens on a carbon atom are replaced with the group ═O, ═S, ═NN(R^{bb})₂, =NNR^{bb}C(═O)R^{aa}—, =NNR^{bb}C(═O)OR^{aa}, =NNR^{bb}S(═O)₂R^{aa}, =NR^{bb}, or =NOR^{cc}; wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R′)₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
   each instance of R^{ee} is, independently, selected from C₁₋₁₀ alkyl, C₁₋₁₀ perhaloalkyl, C₁₋₁₀alkenyl, C₁₋₁₀alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{ff} is, independently, selected from hydrogen, C₁₋₁₀alkyl, C₁₋₁₀ perhaloalkyl, C₁₋₁₀alkenyl, C₁₋₁₀alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or =S; and
   each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}—, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH.

The term “alkoxy” refers to the group —O-(substituted or unsubstituted alkyl).

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{cc}, —SO₂R^{cc}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}—, —C(═NR^{bb})N(R^{bb})₂, —S(═O)Ra, —SO₂R^{aa}, —Si(R^{aa})₃, —P(R^{cc})₂, —P(R^{cc})₃⁺X⁻, —P(OR^{cc})₂, —P(OR)₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆S)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and Claims. The invention is not limited in any manner by the above exemplary listing of substituents.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “crystalline” or “crystalline form” refers to a solid form substantially exhibiting three-dimensional order. In certain embodiments, a crystalline form of a solid is a solid form that is substantially not amorphous. In certain embodiments, the X-ray powder diffraction (XRPD) pattern of a crystalline form includes one or more sharply defined peaks.

The term “amorphous” or “amorphous form” refers to a form of a solid (“solid form”), the form substantially lacking three-dimensional order. In certain embodiments, an amorphous form of a solid is a solid form that is substantially not crystalline. In certain embodiments, the X-ray powder diffraction (XRPD) pattern of an amorphous form includes a wide scattering band with a peak at 2θ of, e.g., between 2° and 70°, inclusive, using CuKα radiation. In certain embodiments, the XRPD pattern of an amorphous form further includes one or more peaks attributed to crystalline structures. In certain embodiments, the maximum intensity of any one of the one or more peaks attributed to crystalline structures observed at a 20 of between 2° and 70°, inclusive, is not more than 300-fold, not more than 100-fold, not more than 30-fold, not more than 10-fold, or not more than 3-fold of the maximum intensity of the wide scattering band. In certain embodiments, the XRPD pattern of an amorphous form includes no peaks attributed to crystalline structures.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. Co-crystals may be useful to improve the properties (e.g., solubility, stability, and ease of formulation) of a compound disclosed herein.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein may be preferred.

The term “isotopically labeled derivative” refers to a compound comprising one or more isotopes. The term “isotopes” refers to variants of a particular chemical element such that, while all isotopes of a given element share the same number of protons in each atom of the element, those isotopes differ in the number of neutrons.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a compound, particle, and/or composition of the disclosure is delivered. A target tissue may be an abnormal or unhealthy tissue, which may need to be treated. A target tissue may also be a normal or healthy tissue that is under a higher than normal risk of becoming abnormal or unhealthy, which may need to be prevented. In certain embodiments, the target tissue is the liver. In certain embodiments, the target tissue is the lung. A “non-target tissue” is any biological tissue of a subject (including a group of cells, a body part, or an organ) or a part thereof, including blood and/or lymph vessels, which is not a target tissue.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound or combination described herein refers to an amount sufficient to elicit the desired biological response. An effective amount of a compound or combination described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the disclosure may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

The term “modulate,” “modulating,” “modulation,” or “modulator” refer to the ability of a compound or combination to reduce/increase, slow/speed up, halt/initiate, inhibit/stimulate, or prevent/cause activity of a particular biological target (e.g., translation of one or more repeat associated non-AUG (RAN) proteins) in a cell relative to vehicle.

As used herein, the term “inhibit” or “inhibition” in the context of translation, for example, in the context of translation of one or more repeat associated non-AUG (RAN) proteins, refers to a reduction in translation. In some embodiments, the term refers to a reduction of the level translation of one or more repeat associated non-AUG (RAN) proteins, to a level that is statistically significantly lower than an initial level, which may, for example, be a baseline level of enzyme activity. In some embodiments, the term refers to a reduction of the level of translation of one or more repeat associated non-AUG (RAN) proteins, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity.

A “therapeutically effective amount” of a compound or combination described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is effective for treating or preventing a disease. In certain embodiments, a therapeutically effective amount is effective for treating a neurological disease associated with repeat expansions. In certain embodiments, a therapeutically effective amount is effective for treating a neurodegenerative disease associated with repeat expansions. In certain embodiments, a therapeutically effective amount is an amount effective in reducing repeat expansions. In certain embodiments, a therapeutically effective amount is an amount effective in reducing the transcription of RNAs that produce RAN proteins. In certain embodiments, a therapeutically effective amount is an amount effective in reducing the translation of RAN proteins. In certain embodiments, a therapeutically effective amount is an amount effective in reducing the level of one or more RAN proteins in a subject. In certain embodiments, a therapeutically effective amount is an amount effective in reducing the level of one or more RAN proteins and treating a neurological disease associated with repeat expansions. In certain embodiments, a therapeutically effective amount is an amount effective in reducing the level of one or more RAN proteins and treating a neurological disease associated with RAN protein accumulation. In certain embodiments, a therapeutically effective amount is an amount effective in reducing the accumulation of RAN proteins. In certain embodiments, a therapeutically effective amount is effective for treating amyotrophic lateral sclerosis (ALS). In certain embodiments, a therapeutically effective amount is effective for treating frontotemporal dementia (FTD). In certain embodiments, a therapeutically effective amount is effective for treating C9ORFf72 ALS. In certain embodiments, a therapeutically effective amount is effective for treating C9ORF72 FTD. In certain embodiments, a therapeutically effective amount is effective for treating spinocerebellar ataxia. In certain embodiments, a therapeutically effective amount is effective for treating spinocerebellar ataxia type 1, spinocerebellar ataxia type 2, spinocerebellar ataxia type 3, or spinocerebellar ataxia type 8. In certain embodiments, a therapeutically effective amount is effective for treating a spinocerebellar ataxia type 6, spinocerebellar ataxia type 7, spinocerebellar ataxia type 10, spinocerebellar ataxia type 12, spinocerebellar ataxia type 17, spinocerebellar ataxia type 31, or spinocerebellar ataxia type 36. In certain embodiments, a therapeutically effective amount is effective for treating myotonic dystrophy. In certain embodiments, a therapeutically effective amount is effective for treating myotonic dystrophy type 1, myotonic dystrophy type 2, or Fuch's corneal endothelial dystrophy. In certain embodiments, a therapeutically effective amount is effective for treating spinal bulbar muscular atrophy. In certain embodiments, a therapeutically effective amount is effective for treating dentatorubral-pallidoluysian atrophy. In certain embodiments, a therapeutically effective amount is effective for treating Huntington's disease. In certain embodiments, a therapeutically effective amount is effective for treating Fragile X Tremor Ataxia Syndrome (FXTAS). In certain embodiments, a therapeutically effective amount is effective for Huntington's disease-like 2 syndrome (HDL2); Fragile X syndrome (FXS); disorders related to 7p11.2 folate-sensitive fragile site FRA7A; disorders related to folate-sensitive fragile site 2q11 FRA2A; or Fragile XE syndrome (FRAXE).

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

A “prophylactically effective amount” of a compound or combination described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is effective for preventing a neurological disease associated with repeat expansions. In certain embodiments, a prophylactically effective amount is effective for preventing a neurological disease associated with RAN protein accumulation. In certain embodiments, a prophylactically effective amount is effective for preventing a neurodegenerative disease associated with repeat expansions. In certain embodiments, a prophylactically effective amount is effective in preventing C9ORFf72 amyotrophic lateral sclerosis (ALS) or C9ORF72 frontotemporal dementia (FTD); Alzheimer's disease; myotonic dystrophy type 1 (DM1) and myotonic dystrophy type 2 (DM2); spinocerebellar ataxia types 1, 2, 3, 6, 7, 8, 10, 12, 17, 31, and 36; spinal bulbar muscular atrophy; dentatorubral-pallidoluysian atrophy (DRPLA); Huntington's disease; Fuch's endothelial corneal dystrophy (FECD); Fragile X Tremor Ataxia Syndrome (FXTAS); Huntington's disease-like 2 syndrome (HDL2); Fragile X syndrome (FXS); disorders related to 7p11.2 folate-sensitive fragile site FRA7A; disorders related to folate-sensitive fragile site 2q11 FRA2A; or Fragile XE syndrome (FRAXE). In certain embodiments, a prophylactically effective amount is effective in reducing the level of RAN proteins in tissues from subjects with gene mutations that can cause C9orf72 amyotrophic lateral sclerosis (ALS); C9orf72 frontotemporal dementia; Alzheimer's disease; myotonic dystrophy type 1 (DM1) and myotonic dystrophy type 2 (DM2); spinocerebellar ataxia types 1, 2, 3, 6, 7, 8, 10, 12, 17, 31, and 36; spinal bulbar muscular atrophy; dentatorubral-pallidoluysian atrophy (DRPLA); Huntington's disease; Fuch's endothelial corneal dystrophy (FECD); Fragile X Tremor Ataxia Syndrome (FXTAS); Huntington's disease-like 2 syndrome (HDL2); Fragile X syndrome (FXS); disorders related to 7p11.2 folate-sensitive fragile site FRA7A; disorders related to folate-sensitive fragile site 2q11 FRA2A; or Fragile XE syndrome (FRAXE). In certain embodiments, a prophylactically effective amount is effective in preventing the accumulation of RAN proteins in tissues from subjects with gene mutations that can cause C9orf72 amyotrophic lateral sclerosis (ALS); C9orf72 frontotemporal dementia; Alzheimer's disease; myotonic dystrophy type 1 (DM1) and myotonic dystrophy type 2 (DM2); spinocerebellar ataxia types 1, 2, 3, 6, 7, 8, 10, 12, 17, 31, and 36; spinal bulbar muscular atrophy; dentatorubral-pallidoluysian atrophy (DRPLA); Huntington's disease; Fuch's endothelial corneal dystrophy (FECD); Fragile X Tremor Ataxia Syndrome (FXTAS)); Huntington's disease-like 2 syndrome (HDL2); Fragile X syndrome (FXS); disorders related to 7p11.2 folate-sensitive fragile site FRA7A; disorders related to folate-sensitive fragile site 2q11 FRA2A; or Fragile XE syndrome (FRAXE).

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, or organelles (such as obtained by lysing cells and separating the components thereof by centrifugation or otherwise). Other examples of biological samples include blood, serum, urine, semen, fecal matter, cerebrospinal fluid, interstitial fluid, mucus, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. Biological samples also include those biological samples that are transgenic, such as transgenic oocyte, sperm cell, blastocyst, embryo, fetus, donor cell, or cell nucleus. Biological samples further include white blood cells in peripheral blood, or brain lysates and cerebrospinal fluid.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine.

A “protein” or “peptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, or synthetic, or any combination of these.

A “RAN protein (repeat-associated non-AUG translated protein)” is a polypeptide translated from sense or antisense RNA sequences carrying a nucleotide expansion without the requirement for an AUG initiation codon. Generally, RAN proteins comprise “expansion repeats” or “repeat expansions” of an amino acid, termed poly amino acid repeats. For example, “AAAAAAAAAAAAAAAAAAAA” (poly-alanine), “LLLLLLLLLLLLLLLLLL” (poly-leucine), “SSSSSSSSSSSSSSSSSSSS” (poly-serine), or “CCCCCCCCCCCCCCCCCCCC” (poly-cysteine) are poly amino acid repeats that are each 20 amino acid residues in length. RAN proteins can have a poly amino acid repeat of at least 25, at least 30, at least 40, at least 50, at least 60, at least 70, at least 80, at least 90, at least 100, or at least 200 amino acid residues in length. In some embodiments, a RAN protein has a poly amino acid repeat more than 200 amino acid residues in length. Generally, RAN proteins are translated from abnormal repeat expansions (e.g., CAG repeats) of DNA. In general, RAN proteins comprise expansion repeats of one or amino acid, termed poly amino acid repeats (e.g., di-amino acid repeats). For example, in the context of ALS/FTD, which results from a repeat expansion of the hexanucleotide sequence GGGGCC in the C9ORF72 gene, the following di-amino acid repeat-containing RAN proteins have been identified: poly-(Gly-Ala), poly-(Gly-Pro), poly-(Gly-Arg), poly-(5 Pro-Ala), or poly-(Pro-Arg), also referred to as poly(GA), poly(GP), poly(GR), poly(PA), and poly(PR), respectively. Without wishing to be bound by any particular theory, RAN protein accumulation (e.g., in the nucleus or cytoplasm of a cell) disrupts cellular function and induces cellular toxicity. In some embodiments, translation and accumulation of RAN proteins is associated with a disease, for example, a neurological disease, neurodegenerative disease, or neurodegenerative disorder. Examples of diseases associated with RAN protein translation and accumulation include but are not limited to C9ORFf72 ALS, C9ORF72 FTD, Alzheimer's disease, myotonic dystrophy type 1 (DM1) and myotonic dystrophy type 2 (DM2), spinocerebellar ataxia types 1, 2, 3, 6, 7, 8, 10, 12, 17, 31, and 36; spinal bulbar muscular atrophy; dentatorubral-pallidoluysian atrophy (DRPLA); Huntington's disease (HD); Fuch's endothelial corneal dystrophy (FECD); Fragile X Tremor Ataxia Syndrome (FXTAS); Huntington's disease-like 2 syndrome (HDL2); Fragile X syndrome (FXS); disorders related to 7p11.2 folate-sensitive fragile site FRA7A; disorders related to folate-sensitive fragile site 2q11 FRA2A; and Fragile XE syndrome (FRAXE).

A “repeat expansion” is a mutation which increases the number of times that a short nucleotide sequence is repeated. Exemplary repeat expansions are provided above in the definition of “RAN protein.”

“C9ORFf72 amyotrophic lateral sclerosis” or “C9ORFf72 ALS” refers to amyotrophic lateral sclerosis associated with a hexanucleotide repeat expansion mutation in the chromosome 9 open reading frame 72 (C9ORFf72) gene. “C9ORF72 frontotemporal dementia” or “C9ORF72 FTD” refers to frontotemporal dementia associated with a hexanucleotide expansion mutation in the C9ORFf72 gene.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells. Examples of neurodegenerative diseases include, but are not limited to, C9ORFf72 ALS, C9ORF72 FTD, Alzheimer's disease, myotonic dystrophy type 1 (DM1) and myotonic dystrophy type 2 (DM2), spinocerebellar ataxia types 1, 2, 3, 6, 7, 8, 10, 12, 17, 31, and 36; spinal bulbar muscular atrophy; dentatorubral-pallidoluysian atrophy (DRPLA); Huntington's disease (HD); Fuch's endothelial corneal dystrophy (FECD); Fragile X Tremor Ataxia Syndrome (FXTAS); Huntington's disease-like 2 syndrome (HDL2); Fragile X syndrome (FXS); disorders related to 7p11.2 folate-sensitive fragile site FRA7A; disorders related to folate-sensitive fragile site 2q11 FRA2A; and Fragile XE syndrome (FRAXE). Neuromuscular diseases refer to a type of neurological disease marked by pathologies of the nerves or neuromuscular junctions. Exemplary neuromuscular diseases include, but are not limited to, amyotrophic lateral sclerosis, multiple sclerosis, and spinal muscular atrophy.

### DETAILED DESCRIPTION OF CERTAIN EMBODIMENTS OF THE INVENTION

The present disclosure provides compositions, methods, uses, and kits comprising a combination of compounds (e.g., metformin, or analog thereof, and a second analog of metformin, or metformin, or analog thereof, and quercetin, or analog thereof). Thus, the compositions, methods, uses, and kits comprise a compound of Formula (I) and either a second compound of Formula (I) or a compound of Formula (II). The compositions, methods, uses, and kits are directed to treating and/or preventing a neurological disease associated with repeat expansions in a subject in need thereof. The combination of compounds is used to inhibit RAN translation. In certain embodiments, the neurological disease to be treated is associated with repeat expansions. In certain embodiments, the neurological disease is associated with repeat expansion mutations that undergo RAN protein translation. In certain embodiments, the neurological disease is associated with the expression of RAN proteins.

Thus, in one aspect, provided herein are methods of treating or preventing a neurological disease associated with repeat expansions in a subject in need thereof, the method comprising administering to the subject: (a) a compound of Formula (I):

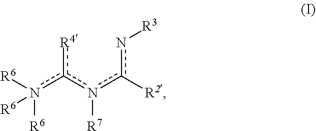

or a pharmaceutically acceptable salt, co-crystal, tautomer, stereoisomer, solvate, hydrate, polymorph, isotopically enriched derivative, or prodrug thereof, wherein [symbol] , R^{2′}, R³, R^{4′}, R⁶, and R⁷ are defined herein; and (b) a second compound of Formula (I), or a pharmaceutically acceptable salt, co-crystal, tautomer, stereoisomer, solvate, hydrate, polymorph, isotopically enriched derivative, or prodrug thereof.

In another aspect, provided herein are methods of treating or preventing a neurological disease associated with repeat expansions in a subject in need thereof, the method comprising administering to the subject:
(a) a compound of Formula (I):

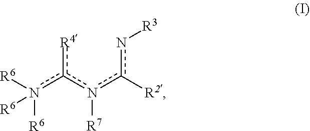

or a pharmaceutically acceptable salt, co-crystal, tautomer, stereoisomer, solvate, hydrate, polymorph, isotopically enriched derivative, or prodrug thereof, wherein [symbol] , R^{2′}, R³, R^{4′}, R⁶, and R⁷ are defined herein; and
(b) a compound of Formula (II):

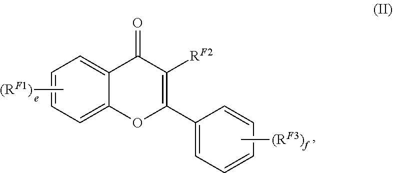

or a pharmaceutically acceptable salt, co-crystal, tautomer, stereoisomer, solvate, hydrate, polymorph, isotopically enriched derivative, or prodrug thereof, wherein R^{F1}, R^{F2}, R^{F3}, e, and f are defined herein.

In another aspect, provided herein are methods of treating or preventing a neurological disease associated with repeat expansions in a subject in need thereof, the method comprising administering to the subject a compound of Formula (II):

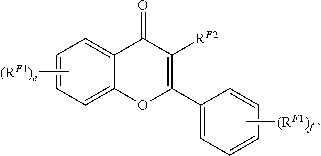

or a pharmaceutically acceptable salt, co-crystal, tautomer, stereoisomer, solvate, hydrate, polymorph, isotopically enriched derivative, or prodrug thereof, wherein R^{F1}, R^{F2}, R^{F3}, e, and f are defined herein.

In yet another aspect, provided herein are pharmaceutical compositions comprising: (a) a compound of Formula (I):

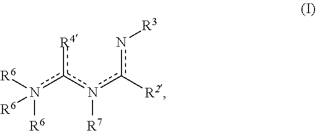

or a pharmaceutically acceptable salt, co-crystal, tautomer, stereoisomer, solvate, hydrate, polymorph, isotopically enriched derivative, or prodrug thereof, wherein [symbol] , R², R³, R⁴, R⁶, and R⁷ are defined herein; and (b) a second compound of Formula (I), or a pharmaceutically acceptable salt, co-crystal, tautomer, stereoisomer, solvate, hydrate, polymorph, isotopically enriched derivative, or prodrug thereof.

In another aspect, provided herein are pharmaceutical compositions comprising: (a) a compound of Formula (I):

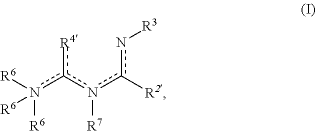

or a pharmaceutically acceptable salt, co-crystal, tautomer, stereoisomer, solvate, hydrate, polymorph, isotopically enriched derivative, or prodrug thereof, wherein [symbol] , R^{2′}, R³, R^{4′}, R⁶, and R⁷ are defined herein; and (b) a compound of Formula (II):

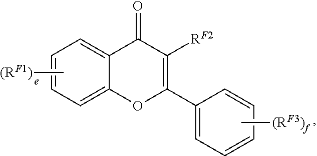

or a pharmaceutically acceptable salt, co-crystal, tautomer, stereoisomer, solvate, hydrate, polymorph, isotopically enriched derivative, or prodrug thereof, wherein R^{F1}, R^{F2}, R^{F3}, e, and f are as defined herein.

### Compounds of Formula (I) and Formula (II)

The methods, uses, compositions, and kits provided herein comprise: (a) two compounds of Formula (I), or (b) a compound of Formula (I) and a compound of Formula (II). When the methods, uses, compositions, and kits comprise two compounds of Formula (I), the first and second compound of Formula (I) are different.

As used herein, a compound of Formula (I) is of the formula:

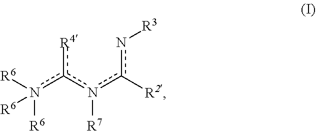

or a pharmaceutically acceptable salt, co-crystal, tautomer, stereoisomer, solvate, hydrate, polymorph, isotopically enriched derivative, or prodrug thereof, wherein:
each instance of [symbol] is a single bond or double bond, as valency permits;
R^{2′} is hydrogen, halogen, or —N(R^{2A})₂;
each instance of R^{2A} is independently hydrogen, optionally substituted acyl, optionally substituted alkyl, or a nitrogen protecting group;
R³ is hydrogen, optionally substituted alkyl, or a nitrogen protecting group;
R^{4′} is hydrogen, —N(R⁴)₂, or

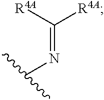

each instance of R⁴ is independently hydrogen, optionally substituted alkyl, a nitrogen protecting group, or absent, as valency permits; or optionally, when R^{4′} is —N(R⁴)₂, one instance of R⁴ is taken together with R³ and the intervening atoms to form an optionally substituted 5 to 7-membered heterocyclic ring;
each instance of R^{4A} is independently hydrogen, optionally substituted alkyl, or a nitrogen protecting group;
each instance of R⁶ is independently hydrogen, optionally substituted alkyl, a nitrogen protecting group, or absent, as valency permits; and
R⁷ is hydrogen, optionally substituted alkyl, a nitrogen protecting group, or absent, as valency permits.

In some embodiments, the compound of Formula (I) is of formula:

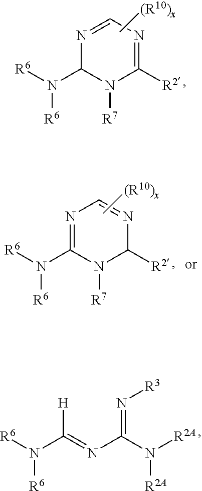

or a pharmaceutically acceptable salt, co-crystal, tautomer, stereoisomer, solvate, hydrate, polymorph, isotopically enriched derivative, or prodrug thereof, wherein:
x is 0 or 1; and
each instance of R¹⁰ is independently halogen, optionally substituted C₁₋₆ alkyl, —NH₂, —NH(optionally substituted C₁₋₆ alkyl), or —N(optionally substituted C₁₋₆ alkyl)₂.

In certain embodiments, the compound of Formula (I) is a compound of Formula (I-A),

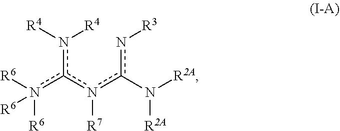

or a pharmaceutically acceptable salt, co-crystal, tautomer, stereoisomer, solvate, hydrate, polymorph, isotopically enriched derivative, or prodrug thereof, wherein:
each instance of [symbol] is a single bond or double bond, as valency permits;
each instance of R^{2A} is independently hydrogen, optionally substituted acyl, optionally substituted alkyl, or a nitrogen protecting group;
R³ is hydrogen, optionally substituted alkyl, or a nitrogen protecting group;
each instance of R⁴ is independently hydrogen, optionally substituted alkyl, a nitrogen protecting group, or absent, as valency permits; or, optionally, one instance of R⁴ is taken together with R³ and the intervening atoms to form an optionally substituted 5 to 7-membered heterocyclic ring;
each instance of R⁶ is independently hydrogen, optionally substituted alkyl, a nitrogen protecting group, or absent, as valency permits; and
R⁷ is hydrogen, optionally substituted alkyl, a nitrogen protecting group, or absent, as valency permits.

In certain embodiments, the compound of Formula (I) is of formula:

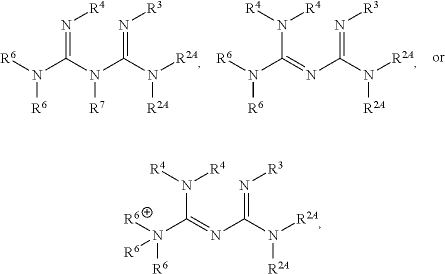

or a pharmaceutically acceptable salt, co-crystal, tautomer, stereoisomer, solvate, hydrate, polymorph, isotopically enriched derivative, or prodrug thereof.

In some embodiments, the compound of Formula (I) is of formula:

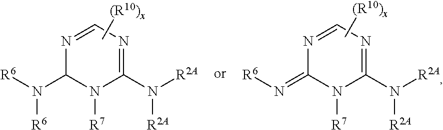

or a pharmaceutically acceptable salt, co-crystal, tautomer, stereoisomer, solvate, hydrate, polymorph, isotopically enriched derivative, or prodrug thereof, wherein:
x is 0 or 1; and
each instance of R¹⁰ is independently halogen, optionally substituted C₁₋₆ alkyl, —NH₂, —NH(optionally substituted C₁₋₆ alkyl), or —N(optionally substituted C₁₋₆ alkyl)₂.

In certain embodiments, the compound of Formula (I) is of Formula (I-B):

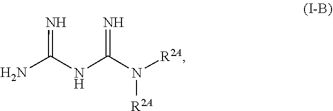

or a pharmaceutically acceptable salt, co-crystal, tautomer, stereoisomer, solvate, hydrate, polymorph, isotopically enriched derivative, or prodrug thereof, wherein:
each instance of R^{2A} is independently hydrogen, optionally substituted alkyl, or a nitrogen protecting group.

In some embodiments, each instance of [symbol] is a single bond or double bond, as valency permits. In some embodiments, some instances of [symbol] is a single bond and some instances of [symbol] is a double bond.

In some embodiments, R^{2′} is hydrogen, halogen, or —N(R^{2A})₂. In some embodiments, R^{2′} is halogen. In certain embodiments, R^{2′} is I. In some embodiments, R^{2′} is —N(R^{2A})₂. In certain embodiments, R^{2′} is —N(R^{2A})₂, and each instance of R^{2A} is independently hydrogen, optionally substituted acyl, optionally substituted alkyl, or a nitrogen protecting group (e.g., —NMe₂). In certain embodiments, R^{2′} is —NMe₂. In certain embodiments, R^{2′} is —N(R^{2A})₂, and each instance of R^{2A} is independently hydrogen or optionally substituted alkyl. In certain embodiments, R^{2′} is —(N¹⁵)(R^{2A})₂, and each instance of R^{2A} is independently hydrogen or optionally substituted alkyl.

Formula (I) includes zero or more instances of substituent R^{2A}. In certain embodiments, each instance of R^{2A} is independently hydrogen, optionally substituted acyl, optionally substituted alkyl, or a nitrogen protecting group. In certain embodiments, at least one instance of R^{2A} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In certain embodiments, at least one instance of R^{2A} is optionally substituted acyl (e.g., —C(═O)Me). In some embodiments, each instance of R^{2A} is independently hydrogen or optionally substituted alkyl. In some embodiments, each instance of R^{2A} is independently hydrogen or optionally substituted C₁₋₆ alkyl. In certain embodiments, at least one instance of R^{2A} is hydrogen. In certain embodiments, both instances of R^{2A} are hydrogen. In certain embodiments, at least one instance of R^{2A} is deuterium. In certain embodiments, both instances of R^{2A} are deuterium. In some embodiments, at least one instance of R^{2A} is optionally substituted C₁₋₆ alkyl. In certain embodiments, at least one instance of R^{2A} is substituted C₁₋₆ alkyl. In certain embodiments, at least one instance of R^{2A} is unsubstituted C₁₋₆ alkyl. In some embodiments, at least one instance of R^{2A} is unsubstituted methyl or unsubstituted ethyl. In certain embodiments, at least one instance of R^{2A} is unsubstituted methyl. In certain embodiments, two instances of R^{2A} are unsubstituted methyl. In some embodiments, at least one instance of R^{2A} is unsubstituted ethyl. In some embodiments, one instance of R^{2A} is hydrogen and one instance of R^{2A} is methyl. In certain embodiments, at least one instance of R^{2A} is unsubstituted ethyl. In certain embodiments, at least one instance of R^{2A} is optionally substituted ethyl. In certain embodiments, at least one instance of R^{2A} is substituted ethyl. In certain embodiments, at least one instance of R^{2A} is of the formula:

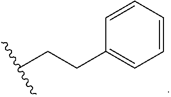 [SMILES: *CCc1ccccc1]

In certain embodiments, at least one instance of R^{2A} is optionally substituted n-propyl. In certain embodiments, at least one instance of R^{2A} is unsubstituted n-propyl.

In some embodiments, R³ is hydrogen, optionally substituted alkyl, or a nitrogen protecting group. In some embodiments, R³ is a nitrogen protecting group. In certain embodiments, R³ is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In some embodiments, R³ is hydrogen or optionally substituted alkyl. In certain embodiments, R³ is hydrogen. In some embodiments, R³ is optionally substituted C₁₋₆ alkyl. In certain embodiments, R³ is unsubstituted methyl or unsubstituted ethyl. In certain embodiments, R³ is optionally substituted ethyl. In certain embodiments, R³ is unsubstituted ethyl. In some embodiments, R³ is substituted methyl. In some embodiments, R³ is unsubstituted methyl.

In certain embodiments, R^{4′} is hydrogen, —N(R⁴)₂, or

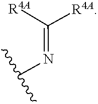

In some embodiments, R^{4′} is hydrogen. In some embodiments, R^{4′} is

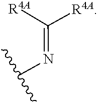

In certain embodiments, R^{4′} is

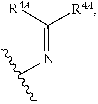

and each instance of R⁴ is independently hydrogen or optionally substituted alkyl. In some embodiments, R^{4′} is —N(R⁴)₂. In certain embodiments, R^{4′} is —N(R⁴)₂, and each instance of R⁴ is independently hydrogen, optionally substituted alkyl, or a nitrogen protecting group, or absent, as valency permits.

In some embodiments, each instance of R⁴ is independently hydrogen, optionally substituted alkyl, a nitrogen protecting group, or absent, as valency permits; or optionally, when R^{4′} is —N(R⁴)₂, one instance of R⁴ is taken together with R³ and the intervening atoms to form an optionally substituted 5 to 7-membered heterocyclic ring. In some embodiments, R⁴ is absent. In certain embodiments, one instance of R⁴ is absent. In certain embodiments, Formula (I) includes two instances of substituent R⁴. In certain embodiments, Formula (I) includes three instances of substituent R⁴. In some embodiments, R⁴ is a nitrogen protecting group. In certain embodiments, at least one instance of R⁴ is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In some embodiments, each instance of R⁴ is independently hydrogen or optionally substituted alkyl. In certain embodiments, at least one instance of R⁴ is hydrogen. In certain embodiments, both instances of R⁴ are hydrogen. In certain embodiments, at least one instance of R⁴ is deuterium. In certain embodiments, both instances of R⁴ are deuterium. In some embodiments, each instance of R⁴ is independently hydrogen or optionally substituted C₁₋₆ alkyl. In some embodiments, at least one instance of R⁴ is optionally substituted C₁₋₆ alkyl. In some embodiments, at least one instance of R⁴ is unsubstituted methyl or unsubstituted ethyl. In certain embodiments, at least one instance of R⁴ is optionally substituted methyl. In some embodiments, at least one instance of R⁴ is unsubstituted methyl. In certain embodiments, two instances of R⁴ are unsubstituted methyl. In some embodiments, at least one instance of R⁴ is unsubstituted ethyl. In some embodiments, one instance of R⁴ is hydrogen, and one instance of R⁴ is methyl.

In some embodiments, one instance of R⁴ is taken together with R³ and the intervening atoms to form an optionally substituted 5 to 7-membered heterocyclic ring. In some embodiments, one instance of R⁴ is taken together with R³ and the intervening atoms to form an optionally substituted 6-membered heterocyclic ring. In some embodiments, one instance of R⁴ is hydrogen or C₁₋₆ alkyl, and one instance of R⁴ is taken together with R³ and the intervening atoms to form an optionally substituted 5 to 7-membered heterocyclic ring.

In some embodiments, each instance of R^{4A} is independently hydrogen, optionally substituted alkyl, or a nitrogen protecting group. In some embodiments, each instance of R^{4A} is a nitrogen protecting group. In certain embodiments, each instance of R^{4A} is independently hydrogen or optionally substituted alkyl. In certain embodiments, each instance of R^{4A} is independently hydrogen or optionally substituted C₁₋₆ alkyl. In certain embodiments, each instance of R^{4A} is hydrogen. In certain embodiments, each instance of R^{4A} is optionally substituted alkyl. In some embodiments, one instance of R⁴ is hydrogen, and one instance of R⁴ is methyl.

In certain embodiments, each instance of R⁶ is independently hydrogen, optionally substituted alkyl, a nitrogen protecting group, or absent, as valency permits. In certain embodiments, R⁶ is absent. In certain embodiments, one instance of R⁶ is absent. In certain embodiments, Formula (I) includes two instances of substituent R⁶. In certain embodiments, Formula (I) includes three instances of substituent R⁶. In some embodiments, R⁶ is a nitrogen protecting group. In certain embodiments, at least one instance of R⁶ is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In some embodiments, R⁶ is independently hydrogen, optionally substituted alkyl, a nitrogen protecting group. In some embodiments, R⁶ is hydrogen or optionally substituted alkyl. In some embodiments, R⁶ is hydrogen or optionally C₁₋₆ substituted alkyl. In some embodiments, at least one instance of R⁶ is optionally substituted C₁₋₆ alkyl. In certain embodiments, R⁶ is hydrogen. In certain embodiments, at least one instance of R⁶ is hydrogen. In certain embodiments, two instances of R⁶ are hydrogen. In certain embodiments, at least one instance of R⁶ is deuterium. In certain embodiments, two instances of R⁶ are deuterium. In some embodiments, R⁶ is optionally substituted C₁₋₆ alkyl. In certain embodiments, at least one instance of R⁶ is optionally substituted alkyl (e.g., substituted or unsubstituted C₁₋₆ alkyl). In certain embodiments, at least one instance of R⁶ is optionally substituted C₁₋₆ alkyl. In certain embodiments, two instances of R⁶ are optionally substituted C₁₋₆ alkyl. In certain embodiments, three instances of R⁶ are optionally substituted C₁₋₆ alkyl, and the moiety:

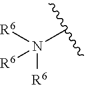

is of the formula:

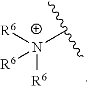

In certain embodiments, at least one instance of R⁶ is unsubstituted C₁₋₆ alkyl. In certain embodiments, R⁶ is unsubstituted methyl or unsubstituted ethyl. In certain embodiments, at least one instance of R⁶ is optionally substituted methyl. In some embodiments, at least one instance of R⁶ is unsubstituted methyl. In some embodiments, R⁶ is unsubstituted methyl. In certain embodiments, at least one instance of R⁶ is unsubstituted ethyl.

In some embodiments, R⁷ is hydrogen, optionally substituted alkyl, a nitrogen protecting group, or absent, as valency permits. In some embodiments, R⁷ is absent. In certain embodiments, R⁷ is hydrogen, optionally substituted alkyl, or a nitrogen protecting group. In certain embodiments, R⁷ is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In some embodiments, R⁷ is hydrogen or optionally substituted alkyl. In some embodiments, R⁷ is hydrogen or optionally substituted C₁₋₆ alkyl. In certain embodiments, R⁷ is hydrogen. In certain embodiments, R⁷ is deuterium. In some embodiments, R⁷ is optionally substituted C₁₋₆ alkyl. In certain embodiments, R⁷ is unsubstituted methyl or unsubstituted ethyl. In certain embodiments, R⁷ is optionally substituted methyl. In some embodiments, R⁷ is unsubstituted methyl. In certain embodiments, R⁷ is unsubstituted ethyl.

In some embodiments, x is 0 or 1. In certain embodiments, x is 0. In some embodiments, x is 1.

In some embodiments, R¹⁰ is independently halogen, optionally substituted C₁₋₆ alkyl, —NH₂, —NH(optionally substituted C₁₋₆ alkyl), or —N(optionally substituted C₁₋₆ alkyl)₂. In some embodiments, R¹⁰ is halogen. In certain embodiments, at least one instance of R¹⁰ is I. In certain embodiments, R¹⁰ is optionally substituted C₁₋₆ alkyl, —NH₂, —NH(optionally substituted C₁₋₆ alkyl), or —N(optionally substituted C₁₋₆ alkyl)₂. In certain embodiments, R¹⁰ is optionally substituted C₁₋₆ alkyl. In certain embodiments, R¹⁰ is methyl or ethyl. In some embodiments, R¹⁰ is methyl. In some embodiments, at least one instance of R¹⁰ is optionally substituted C₁₋₆ alkyl. In certain embodiments, at least one instance of R¹⁰ is optionally substituted methyl. In certain embodiments, at least one instance of R¹⁰ is optionally substituted ethyl. In certain embodiments, R¹⁰ is —NH₂, —NH(optionally substituted C₁₋₆ alkyl), or —N(optionally substituted C₁₋₆ alkyl)₂. In certain embodiments, at least one instance of R¹⁰ is —NH₂. In certain embodiments, at least one instance of R¹⁰ is —N(optionally substituted alkyl)₂ (e.g., —N(substituted or unsubstituted C₁₋₆ alkyl)₂). In certain embodiments, at least one instance of R¹⁰ is —NH(optionally substituted alkyl)₂ (e.g., —NH(substituted or unsubstituted C₁₋₆ alkyl).

In some embodiments, the compound of Formula (I) is of formula:

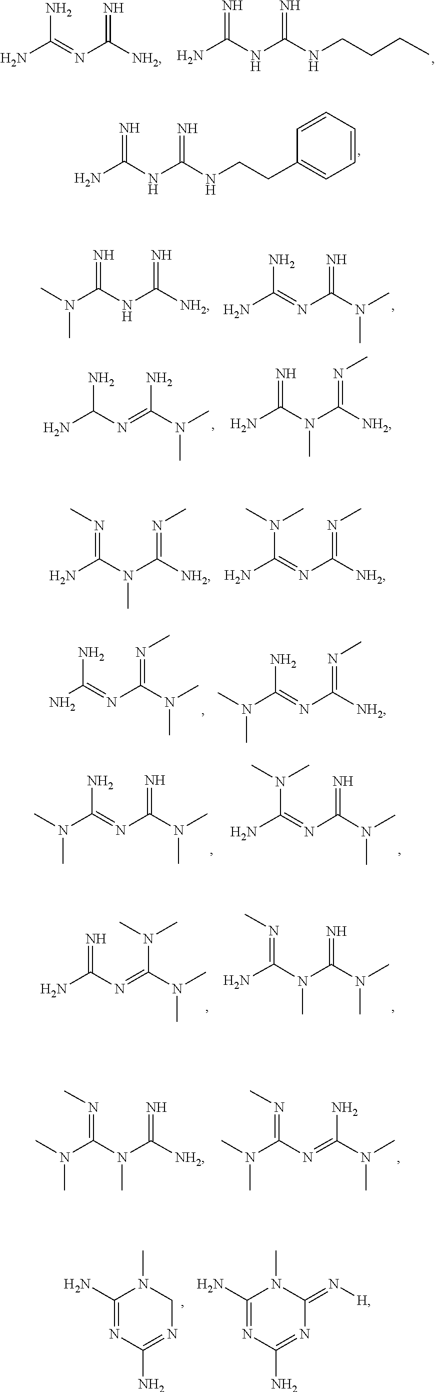

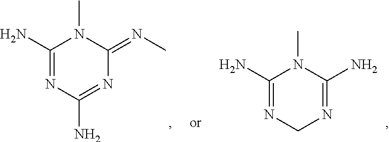

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of formula:

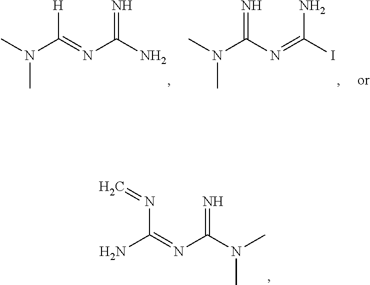

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of formula:

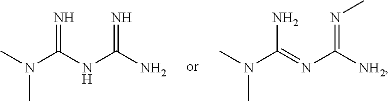

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of formula:

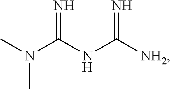

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of formula:

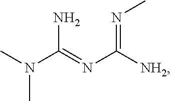

or a pharmaceutically acceptable salt, co-crystal, tautomer, stereoisomer, solvate, hydrate, polymorph, isotopically enriched derivative, or prodrug thereof.

The methods, compositions, and kits provided herein optionally comprise a compound of Formula (II), as well as a compound of Formula (I) as described above.

As used herein, a compound of Formula (II) is of the formula:

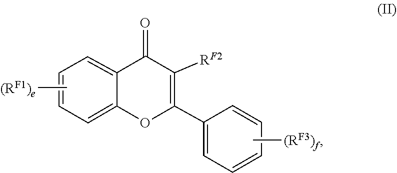

or a pharmaceutically acceptable salt, co-crystal, tautomer, stereoisomer, solvate, hydrate, polymorph, isotopically enriched derivative, or prodrug thereof, wherein:
each instance of R^{F1} is independently hydrogen, hydroxy, or alkoxy;
R^{F2} is hydrogen, hydroxy, or alkoxy;
each instance of R^{F3} is independently hydrogen, hydroxy, or alkoxy;
e is an integer selected from the group consisting of 0, 1, 2, 3, and 4; and
f is an integer selected from the group consisting of 0, 1, 2, 3, 4, and 5.

In some embodiments, each instance of R^{F1} is independently hydrogen, hydroxy, or methoxy; R^{F2} is hydrogen or hydroxy; and each instance of R^{F3} is independently hydrogen, hydroxy, or methoxy.

In some embodiments, each instance of R^{F1} is independently hydrogen, hydroxy, or alkoxy. In certain embodiments, each instance of R^{F1} is independently hydrogen, hydroxy, methoxy, or ethoxy. In some embodiments, each instance of R^{F1} is independently hydrogen, hydroxy, or methoxy. In some embodiments, each instance of R^{F1} is independently hydroxy or methoxy. In certain embodiments, each instance of R^{F1} is methoxy. In certain embodiments, each instance of R^{F1} is hydroxy.

In certain embodiments, e is 0. In some embodiments, e is 1. In some embodiments, e is 2. In some embodiments, e is 3. In certain embodiments, e is 4. In some embodiments, e is preferably 2.

In some embodiments, e is 2, and R^{F1} is hydroxy.

In some embodiments, R^{F2} is hydrogen, hydroxy, or alkoxy. In some embodiments, R^{F2} is hydrogen. In some embodiments, R^{F2} is hydroxy.

In some embodiments, each instance of R^{F3} is independently hydrogen, hydroxy, or alkoxy. In certain embodiments, each instance of R^{F3} is independently hydrogen, hydroxy, methoxy, or ethoxy. In some embodiments, each instance of R^{F3} is independently hydrogen, hydroxy, or methoxy. In some embodiments, each instance of R^{F3} is independently hydroxy or methoxy. In certain embodiments, each instance of R^{F3} is methoxy. In certain embodiments, each instance of R^{F3} is hydroxy.

In certain embodiments, f is 0. In some embodiments, f is 1. In some embodiments, f is 2. In some embodiments, f is 3. In certain embodiments, f is 4. In some embodiments, f is 5. In some embodiments, f is preferably 2.

In some embodiments, f is 2, and R^{F3} is hydroxy.

In certain embodiments, the compound of Formula (II) is of Formula (II-A):

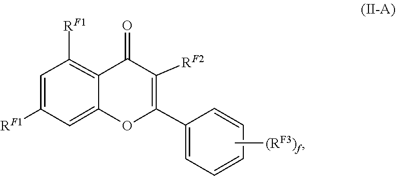

or a pharmaceutically acceptable salt, co-crystal, tautomer, stereoisomer, solvate, hydrate, polymorph, isotopically enriched derivative, or prodrug thereof,

In certain embodiments, the compound of Formula (II) is of Formula (II-B):

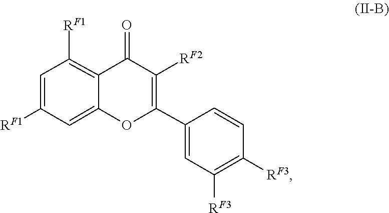

or a pharmaceutically acceptable salt, co-crystal, tautomer, stereoisomer, solvate, hydrate, polymorph, isotopically enriched derivative, or prodrug thereof,

In certain embodiments, the compound of Formula (II) is of formula:

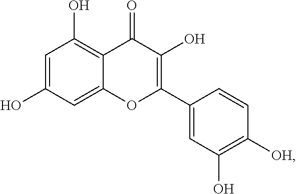 [SMILES: O=c1c(O)c(-c2ccc(O)c(O)c2)oc2cc(O)cc(O)c12]

or a pharmaceutically acceptable salt, co-crystal, tautomer, stereoisomer, solvate, hydrate, polymorph, isotopically enriched derivative, or prodrug thereof.

In some embodiments, the compound of Formula (II) is of formula:

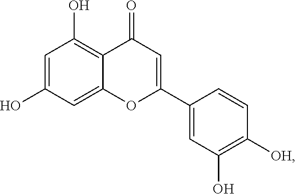 [SMILES: O=c1cc(-c2ccc(O)c(O)c2)oc2cc(O)cc(O)c12]

or a pharmaceutically acceptable salt, co-crystal, tautomer, stereoisomer, solvate, hydrate, polymorph, isotopically enriched derivative, or prodrug thereof.

The compounds provided herein (i.e., compounds of Formulae (I) and (II)) may be in the form of the compound, or a pharmaceutically acceptable salt, co-crystal, tautomer, stereoisomer, solvate, hydrate, polymorph, isotopically enriched derivative, or prodrug thereof. The compounds provided herein (i.e., compounds of Formulae (I) and (II)) may be in the form of the compound, or pharmaceutically acceptable salt, tautomer, solvate, hydrate, isotopically enriched derivative, or prodrug thereof. The compounds provided herein (i.e., compounds of Formulae (I) and (II)) may be in the form of the compound, or a pharmaceutically acceptable salt or tautomer thereof.

### Methods

Provided herein are methods comprising administering a compound of Formula (I), or a pharmaceutically acceptable salt, co-crystal, tautomer, stereoisomer, solvate, hydrate, polymorph, isotopically enriched derivative, or prodrug thereof, and either a second compound of Formula (I), or a pharmaceutically acceptable salt, co-crystal, tautomer, stereoisomer, solvate, hydrate, polymorph, isotopically enriched derivative, or prodrug thereof, or a compound of Formula (II), or a pharmaceutically acceptable salt, co-crystal, tautomer, stereoisomer, solvate, hydrate, polymorph, isotopically enriched derivative, or prodrug thereof.

Also provided herein are methods comprising administering a compound of Formula (II), or a pharmaceutically acceptable salt, co-crystal, tautomer, stereoisomer, solvate, hydrate, polymorph, isotopically enriched derivative, or prodrug thereof.

In certain embodiments, provided herein are methods of treating or preventing a neurological disease associated with repeat expansions in a subject in need thereof, the method comprising administering to the subject a compound of Formula (II):

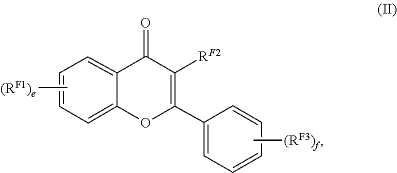

or a pharmaceutically acceptable salt, co-crystal, tautomer, stereoisomer, solvate, hydrate, polymorph, isotopically enriched derivative, or prodrug thereof, wherein:
each instance of R^{F1} is independently hydrogen, hydroxy, or alkoxy;
R^{F2} is hydrogen, hydroxy, or alkoxy;
each instance of R^{F3} is independently hydrogen, hydroxy, or alkoxy;
e is an integer selected from the group consisting of 0, 1, 2, 3, and 4; and
f is an integer selected from the group consisting of 0, 1, 2, 3, 4, and 5.

In certain embodiments, provided herein are methods of treating a neurological disease associated with repeat expansions in a subject in need thereof, the method comprising administering to the subject: (a) a compound of Formula (I):

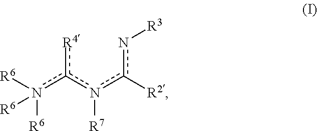

or a pharmaceutically acceptable salt, co-crystal, tautomer, stereoisomer, solvate, hydrate, polymorph, isotopically enriched derivative, or prodrug thereof, wherein [symbol] , R^{2′}, R³, R^{4′}, R⁶, and R⁷ are defined herein; and (b) a second compound of Formula (I), or a pharmaceutically acceptable salt, co-crystal, tautomer, stereoisomer, solvate, hydrate, polymorph, isotopically enriched derivative, or prodrug thereof.

In some embodiments, provided herein are methods of treating a neurological disease associated with repeat expansions in a subject in need thereof, the method comprising administering to the subject: (a) a compound of Formula (I):

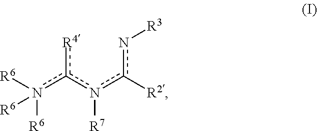

or a pharmaceutically acceptable salt, co-crystal, tautomer, stereoisomer, solvate, hydrate, polymorph, isotopically enriched derivative, or prodrug thereof, wherein [symbol] , R^{2′}, R³, R^{4′}, R⁶, and R⁷ are defined herein; and (b) a compound of Formula (II):

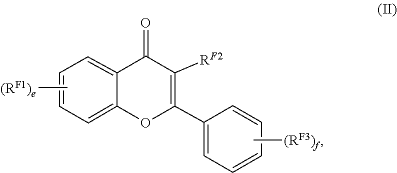

or a pharmaceutically acceptable salt, co-crystal, tautomer, stereoisomer, solvate, hydrate, polymorph, isotopically enriched derivative, or prodrug thereof, wherein R^{F1}, R^{F2}, R^{F3}, e, and f are as defined herein.

In certain embodiments, provided herein are methods of preventing a neurological disease associated with repeat expansions in a subject in need thereof, the method comprising administering to the subject: (a) a compound of Formula (I):

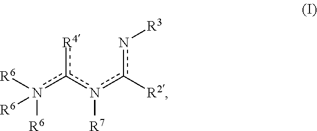

or a pharmaceutically acceptable salt, co-crystal, tautomer, stereoisomer, solvate, hydrate, polymorph, isotopically enriched derivative, or prodrug thereof, wherein [symbol] , R^{2′}, R³, R^{4′}, R⁶, and R⁷ are defined herein; and (b) a second compound of Formula (I), or a pharmaceutically acceptable salt, co-crystal, tautomer, stereoisomer, solvate, hydrate, polymorph, isotopically enriched derivative, or prodrug thereof.

In some embodiments, provided herein are methods of treating or preventing a neurological disease associated with repeat expansions in a subject in need thereof, the method comprising administering to the subject: (a) a compound of Formula (I):

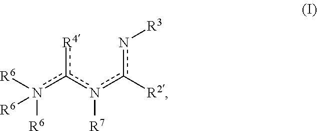

or a pharmaceutically acceptable salt, co-crystal, tautomer, stereoisomer, solvate, hydrate, polymorph, isotopically enriched derivative, or prodrug thereof, wherein [symbol] , R^{2′}, R³, R^{4′}, R⁶, and R⁷ are defined herein; and (b) a compound of Formula (II):

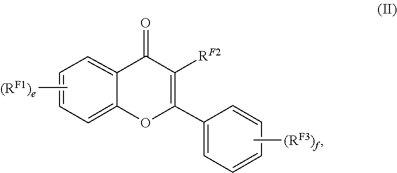

or a pharmaceutically acceptable salt, co-crystal, tautomer, stereoisomer, solvate, hydrate, polymorph, isotopically enriched derivative, or prodrug thereof, wherein R^{F1}, R^{F2}, R^{F3}, e, and f are as defined herein.

In some embodiments, the methods provided herein comprise administering (a) a compound of Formula (I):

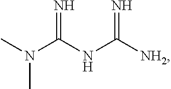

or a pharmaceutically acceptable salt, co-crystal, tautomer, stereoisomer, solvate, hydrate, polymorph, isotopically enriched derivative, or prodrug thereof; and (b) a compound of Formula (II):

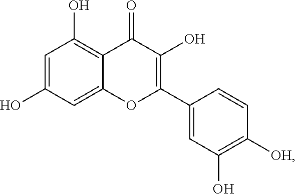 [SMILES: O=c1c(O)c(-c2ccc(O)c(O)c2)oc2cc(O)cc(O)c12]

or a pharmaceutically acceptable salt, co-crystal, tautomer, stereoisomer, solvate, hydrate, polymorph, isotopically enriched derivative, or prodrug thereof. In some embodiments, the methods provided herein comprise administering: (a) a compound of Formula (I):

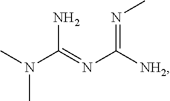

or a pharmaceutically acceptable salt, co-crystal, tautomer, stereoisomer, solvate, hydrate, polymorph, isotopically enriched derivative, or prodrug thereof; and (b) a compound of Formula (II):

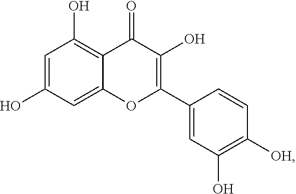 [SMILES: O=c1c(O)c(-c2ccc(O)c(O)c2)oc2cc(O)cc(O)c12]

or a pharmaceutically acceptable salt, co-crystal, tautomer, stereoisomer, solvate, hydrate, polymorph, isotopically enriched derivative, or prodrug thereof. In certain embodiments, the methods provided herein comprise administering: (a) a compound of Formula (I):

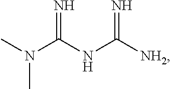

or a pharmaceutically acceptable salt, co-crystal, tautomer, stereoisomer, solvate, hydrate, polymorph, isotopically enriched derivative, or prodrug thereof; and (b) a compound of Formula (II):

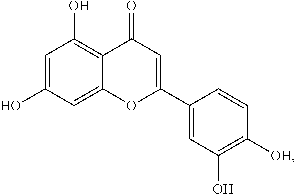 [SMILES: O=c1cc(-c2ccc(O)c(O)c2)oc2cc(O)cc(O)c12]

or a pharmaceutically acceptable salt, co-crystal, tautomer, stereoisomer, solvate, hydrate, polymorph, isotopically enriched derivative, or prodrug thereof. In some embodiments, the methods provided herein comprise administering (a) a compound of Formula (I):

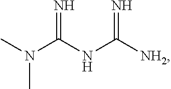

or a pharmaceutically acceptable salt, co-crystal, tautomer, stereoisomer, solvate, hydrate, polymorph, isotopically enriched derivative, or prodrug thereof; and (b) a second compound of Formula (I):

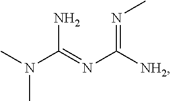

or a pharmaceutically acceptable salt, co-crystal, tautomer, stereoisomer, solvate, hydrate, polymorph, isotopically enriched derivative, or prodrug thereof.

In some embodiments, the methods provided herein comprise a compound of Formula (II):

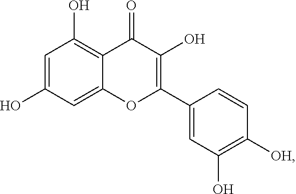 [SMILES: O=c1c(O)c(-c2ccc(O)c(O)c2)oc2cc(O)cc(O)c12]

or a pharmaceutically acceptable salt, co-crystal, tautomer, stereoisomer, solvate, hydrate, polymorph, isotopically enriched derivative, or prodrug thereof. In certain embodiments, the methods provided herein comprise administering a compound of Formula (II):

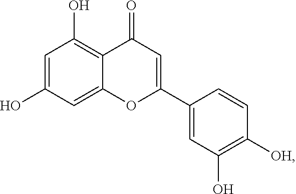 [SMILES: O=c1cc(-c2ccc(O)c(O)c2)oc2cc(O)cc(O)c12]

or a pharmaceutically acceptable salt, co-crystal, tautomer, stereoisomer, solvate, hydrate, polymorph, isotopically enriched derivative, or prodrug thereof.

In one aspect, the disclosure provides methods for administering to a subject a therapeutically effective amount of a compound of Formula (I), or a pharmaceutically acceptable salt, co-crystal, tautomer, stereoisomer, solvate, hydrate, polymorph, isotopically enriched derivative, or prodrug thereof, and either a second compound of Formula (I), or a pharmaceutically acceptable salt, co-crystal, tautomer, stereoisomer, solvate, hydrate, polymorph, isotopically enriched derivative, or prodrug thereof, or a compound of Formula (II), or a pharmaceutically acceptable salt, co-crystal, tautomer, stereoisomer, solvate, hydrate, polymorph, isotopically enriched derivative, or prodrug thereof. In one aspect, the disclosure provides methods for contacting to the biological sample (e.g., cells or tissue) a therapeutically effective amount of a compound of Formula (I), or a pharmaceutically acceptable salt, co-crystal, tautomer, stereoisomer, solvate, hydrate, polymorph, isotopically enriched derivative, or prodrug thereof, and either a second compound of Formula (I), or a pharmaceutically acceptable salt, co-crystal, tautomer, stereoisomer, solvate, hydrate, polymorph, isotopically enriched derivative, or prodrug thereof, or a compound of Formula (II), or a pharmaceutically acceptable salt, co-crystal, tautomer, stereoisomer, solvate, hydrate, polymorph, isotopically enriched derivative, or prodrug thereof. In one aspect, the disclosure provides methods for administering to a subject a therapeutically effective amount of a compound of Formula (II), or a pharmaceutically acceptable salt, co-crystal, tautomer, stereoisomer, solvate, hydrate, polymorph, isotopically enriched derivative, or prodrug thereof. In one aspect, the disclosure provides methods for contacting to a biological sample (e.g., cells or tissue) a therapeutically effective amount of a compound of Formula (II), or a pharmaceutically acceptable salt, co-crystal, tautomer, stereoisomer, solvate, hydrate, polymorph, isotopically enriched derivative, or prodrug thereof. Biological samples include, but are not limited to, cells, tissue, organ, cerebrospinal fluid, blood, or tissue biopsy samples from a subject.

In certain embodiments, the disclosure provides a methods for treating and/or preventing a neurological disease associated with repeat expansions in a subject, the method comprising administering to the subject a therapeutically effective amount of a compound of Formula (I), or a pharmaceutically acceptable salt, co-crystal, tautomer, stereoisomer, solvate, hydrate, polymorph, isotopically enriched derivative, or prodrug thereof, and either a second compound of Formula (I), or a pharmaceutically acceptable salt, co-crystal, tautomer, stereoisomer, solvate, hydrate, polymorph, isotopically enriched derivative, or prodrug thereof; or a compound of Formula (II), or a pharmaceutically acceptable salt, co-crystal, tautomer, stereoisomer, solvate, hydrate, polymorph, isotopically enriched derivative, or prodrug thereof. In certain embodiments, provided herein are methods for treating and/or preventing a neurological disease associated with repeat expansions in a biological sample (e.g., cells or tissue (e.g. from a patient with the disease)), the method comprising contacting the biological sample with a therapeutically effective amount of a compound of Formula (I), or a pharmaceutically acceptable salt, co-crystal, tautomer, stereoisomer, solvate, hydrate, polymorph, isotopically enriched derivative, or prodrug thereof, and either a second compound of Formula (I), or a pharmaceutically acceptable salt, co-crystal, tautomer, stereoisomer, solvate, hydrate, polymorph, isotopically enriched derivative, or prodrug thereof; or a compound of Formula (II), or a pharmaceutically acceptable salt, co-crystal, tautomer, stereoisomer, solvate, hydrate, polymorph, isotopically enriched derivative, or prodrug thereof. Another aspect of the disclosure relates to methods for treating and/or preventing a neurological disease associated with repeat expansions in a subject or cell, by administering to the subject or contacting the biological sample (e.g., cells or tissue) with a therapeutically effective amount of a compound of Formula (I), or a pharmaceutically acceptable salt, co-crystal, tautomer, stereoisomer, solvate, hydrate, polymorph, isotopically enriched derivative, or prodrug thereof, and either a second compound of Formula (I), or a pharmaceutically acceptable salt, co-crystal, tautomer, stereoisomer, solvate, hydrate, polymorph, isotopically enriched derivative, or prodrug thereof, or a compound of Formula (II), or a pharmaceutically acceptable salt, co-crystal, tautomer, stereoisomer, solvate, hydrate, polymorph, isotopically enriched derivative, or prodrug thereof, whereby the method comprises modulating RAN protein translation. In some embodiments, the modulating is inhibiting RAN protein translation. In certain embodiments, the modulating is decreasing RAN protein translation. In some embodiments, the method treats a neurological disease in a subject. In certain embodiments, the method prevents a neurological disease in a subject.

In certain embodiments, the disclosure provides methods for treating and/or preventing a neurological disease associated with repeat expansions in a subject, the method comprising administering to the subject a therapeutically effective amount of or a compound of Formula (II), or a pharmaceutically acceptable salt, co-crystal, tautomer, stereoisomer, solvate, hydrate, polymorph, isotopically enriched derivative, or prodrug thereof. In certain embodiments, provided herein are methods for treating and/or preventing a neurological disease associated with repeat expansions in a biological sample (e.g., cells or tissue (e.g. from a patient with the disease)), the method comprising contacting the biological sample with a therapeutically effective amount of a compound of Formula (II), or a pharmaceutically acceptable salt, co-crystal, tautomer, stereoisomer, solvate, hydrate, polymorph, isotopically enriched derivative, or prodrug thereof. Another aspect of the disclosure relates to methods for treating and/or preventing a neurological disease associated with repeat expansions in a subject or cell, by administering to the subject or contacting the biological sample (e.g., cells or tissue) with a therapeutically effective amount of a compound of Formula (II), or a pharmaceutically acceptable salt, co-crystal, tautomer, stereoisomer, solvate, hydrate, polymorph, isotopically enriched derivative, or prodrug thereof, whereby the method comprises modulating RAN protein translation. In some embodiments, the modulating is inhibiting RAN protein translation. In certain embodiments, the modulating is decreasing RAN protein translation. In some embodiments, the method treats a neurological disease in a subject. In certain embodiments, the method prevents a neurological disease in a subject.

The present disclosure also provides methods of treating and/or preventing a neurological disease associated with RAN protein accumulation in a subject in need thereof of, the method comprising administering to the subject a therapeutically effective amount of a compound of Formula (I), or a pharmaceutically acceptable salt, co-crystal, tautomer, stereoisomer, solvate, hydrate, polymorph, isotopically enriched derivative, or prodrug thereof; and either a second compound of Formula (I), or a pharmaceutically acceptable salt, co-crystal, tautomer, stereoisomer, solvate, hydrate, polymorph, isotopically enriched derivative, or prodrug thereof, or a compound of Formula (II), or a pharmaceutically acceptable salt, co-crystal, tautomer, stereoisomer, solvate, hydrate, polymorph, isotopically enriched derivative, or prodrug thereof. In some embodiments, the method treats a neurological disease in a subject. In certain embodiments, the method prevents a neurological disease in a subject.

The present disclosure also provides methods of treating and/or preventing a neurological disease associated with RAN protein accumulation in a subject in need thereof of, the method comprising administering to the subject a therapeutically effective amount of a compound of Formula (II), or a pharmaceutically acceptable salt, co-crystal, tautomer, stereoisomer, solvate, hydrate, polymorph, isotopically enriched derivative, or prodrug thereof. In some embodiments, the method treats a neurological disease in a subject. In certain embodiments, the method prevents a neurological disease in a subject.

Another aspect of the disclosure relates to methods of modulating RAN protein translation in a subject or cell, the method comprising administering to the subject or contacting the biological sample (e.g., cells or tissue) with a therapeutically effective amount of a compound of Formula (I), or a pharmaceutically acceptable salt, co-crystal, tautomer, stereoisomer, solvate, hydrate, polymorph, isotopically enriched derivative, or prodrug thereof, and either a second compound of Formula (I), or a pharmaceutically acceptable salt, co-crystal, tautomer, stereoisomer, solvate, hydrate, polymorph, isotopically enriched derivative, or prodrug thereof, or a compound of Formula (II), or a pharmaceutically acceptable salt, co-crystal, tautomer, stereoisomer, solvate, hydrate, polymorph, isotopically enriched derivative, or prodrug thereof. Another aspect of the disclosure relates to methods of modulating RAN protein translation in a subject or cell, the method comprising administering to the subject or contacting the biological sample (e.g., cells or tissue) with a therapeutically effective amount of a compound of Formula (II), or a pharmaceutically acceptable salt, co-crystal, tautomer, stereoisomer, solvate, hydrate, polymorph, isotopically enriched derivative, or prodrug thereof. In some embodiments, the modulating is inhibiting RAN protein translation. In certain embodiments, the modulating is decreasing RAN protein translation. In some embodiments, the modulation reduces the level of RAN proteins. In certain embodiments, the method comprises modulating the steady state levels of RAN proteins. In certain embodiments, the method comprises reducing the accumulation of RAN protein in a subject. In certain embodiments, the method comprises reducing the accumulation of RAN protein in a tissue. In certain embodiments, the method comprises reducing the accumulation of RAN protein in a cell. In certain embodiments, the modulating comprises negative regulation of RAN protein translation. In certain embodiments, the modulating comprises inhibition of RAN protein translation. In certain embodiments, the modulating comprises negative regulation of RAN protein translation and reduced accumulation of RAN protein in a cell. In certain embodiments, the modulating comprises negative regulation of RAN protein accumulation in a cell or in patient tissue. In certain embodiments, the modulating comprises changes related to translation of RAN proteins. In certain embodiments, the modulating comprises changes related to turnover of RAN proteins.

In certain embodiments, the disclosure relates to methods of reducing the level of one or more RAN proteins in a biological sample (e.g., cell or tissue) or subject by administering to the subject or contacting the biological sample with a compound of Formula (I), or a pharmaceutically acceptable salt, co-crystal, tautomer, stereoisomer, solvate, hydrate, polymorph, isotopically enriched derivative, or prodrug thereof; and either a second compound of Formula (I), or a pharmaceutically acceptable salt, co-crystal, tautomer, stereoisomer, solvate, hydrate, polymorph, isotopically enriched derivative, or prodrug thereof, or a compound of Formula (II), or a pharmaceutically acceptable salt, co-crystal, tautomer, stereoisomer, solvate, hydrate, polymorph, isotopically enriched derivative, or prodrug thereof. The level of any RAN protein may be reduced using a compound of Formula (I), or a pharmaceutically acceptable salt, co-crystal, tautomer, stereoisomer, solvate, hydrate, polymorph, isotopically enriched derivative, or prodrug thereof; and either a second compound of Formula (I), or a pharmaceutically acceptable salt, co-crystal, tautomer, stereoisomer, solvate, hydrate, polymorph, isotopically enriched derivative, or prodrug thereof, or a compound of Formula (II), or a pharmaceutically acceptable salt, co-crystal, tautomer, stereoisomer, solvate, hydrate, polymorph, isotopically enriched derivative, or prodrug thereof. In certain embodiments, the disclosure relates to methods of reducing the level of one or more RAN proteins in a biological sample (e.g., cell or tissue) or subject by administering to the subject or contacting the biological sample with a compound of Formula (II), or a pharmaceutically acceptable salt, co-crystal, tautomer, stereoisomer, solvate, hydrate, polymorph, isotopically enriched derivative, or prodrug thereof. The level of any RAN protein may be reduced using a compound of Formula (II), or a pharmaceutically acceptable salt, co-crystal, tautomer, stereoisomer, solvate, hydrate, polymorph, isotopically enriched derivative, or prodrug thereof. The level could be the level of any one type of RAN protein, multiple types of RAN proteins, or all RAN proteins.

Another aspect of the disclosure relates to methods of reducing the accumulation of repeat associated non-AUG (RAN) protein in a subject or biological sample (e.g., cell or tissue), the method comprising administering to the subject or biological sample a therapeutically effective amount a compound of Formula (I), or a pharmaceutically acceptable salt, co-crystal, tautomer, stereoisomer, solvate, hydrate, polymorph, isotopically enriched derivative, or prodrug thereof; and either a second compound of Formula (I), or a pharmaceutically acceptable salt, co-crystal, tautomer, stereoisomer, solvate, hydrate, polymorph, isotopically enriched derivative, or prodrug thereof, or a compound of Formula (II), or a pharmaceutically acceptable salt, co-crystal, tautomer, stereoisomer, solvate, hydrate, polymorph, isotopically enriched derivative, or prodrug thereof. Another aspect of the disclosure relates to methods of reducing the accumulation of repeat associated non-AUG (RAN) protein in a subject or biological sample (e.g., cell or tissue), the method comprising administering to the subject or biological sample a therapeutically effective amount a compound of Formula (II), or a pharmaceutically acceptable salt, co-crystal, tautomer, stereoisomer, solvate, hydrate, polymorph, isotopically enriched derivative, or prodrug thereof.

In some embodiments, the method reduces the level of one or more RAN proteins in the subject. In certain embodiments, the method reduces the accumulation of one or more RAN proteins in the subject. In some embodiments, the method reduces the translation of one or more RAN proteins. In certain embodiments, the method inhibits the translation of one or more RAN proteins.

In certain embodiments, the one or more RAN proteins are selected from the group consisting of poly(Alanine) [polyAla or poly(A)]; poly(Arginine) [polyArg or poly(R)]; poly(Arginine-Glutamate) [poly(RE)]; poly(Cysteine) [polyCys or poly(C)]; poly(Cysteine-Proline) [poly(CP)]; poly(Glutamine) [polyGln or poly(Q)]; poly(Glutamine-Alanine) [poly(QA)]; poly(Glycine) [poly(G)]; poly(Glycine-Alanine) [poly(GA)]; poly(Glycine-Arginine) [poly(GR)]; poly(Glycine-Aspartate) [poly(GD)]; poly(Glycine-Glutamate) [poly(GE)]; poly(Glycine-Glutamine) [poly(GQ)]; poly(Glycine-Leucine) [poly(GL)]; poly(Glycine-Lysine) [poly(GK)]; poly(Glycine-Proline) [(poly(GP)]; poly(Glycine-Threonine) [poly(GT)]; poly(Leucine) [polyLeu or poly(L)]; poly(Leucine-Proline) [poly(LP)]; poly(Leucine-Serine) [poly(LS)]; poly(phenylalanine-proline) [poly(FP)]; poly(Proline) [poly(P)]; poly(Proline-Alanine) [poly(PA)]; poly(Proline-Arginine) [poly(PR)]; poly(Serine) [polySer]; poly(Serine-Proline) [poly(SP)]; poly(Tryptophan-Alanine) [poly(WA)]; poly(Valine-Proline) [poly(VP)]; poly(Leucine-Proline-Alanine-Cysteine) [poly(LPAC)]; poly(Glutamine-Alanine-Glycine-Arginine) [poly(QAGR)]; Poly(Isoleucine-Leucine-Phenylalanine-Tyrosine-Serine) [poly(ILFTS)]; Poly(Phenylalanine-Histidine-Serine-Isoleucine-Proline) [poly(FHSIP)]; Poly(Tryptophan-Asparagine-Glycine-Methionine-Glutamine) [poly(WNGMQ)]; poly(PGGRGE); poly(FTPLSLPV); poly(LLPSPSRC); poly(YSPLPPGV); poly(HREGEGSK); poly(TGRERGVN); poly(GRQRGVNT); and poly(GSKHREAE), and combinations thereof. In some embodiments, the RAN protein is poly(Alanine) [polyAla or poly(A)]. In some embodiments, the RAN protein is poly(Arginine) [polyArg or poly(R)]. In some embodiments, the RAN protein is poly(Arginine-Glutamate) [poly(RE)]. In some embodiments, the RAN protein is poly(Cysteine) [polyCys or poly(C)]. In some embodiments, the RAN protein is poly(Cysteine-Proline) [poly(CP)]. In some embodiments, the RAN protein is poly(Glutamine) [polyGln or poly(Q)]. In some embodiments, the RAN protein is poly(Glutamine-Alanine) [poly(QA)]. In some embodiments, the RAN protein is poly(Glycine) [poly(G)]. In some embodiments, the RAN protein is poly(Glycine-Alanine) [poly(GA)]. In some embodiments, the RAN protein is poly(Glycine-Arginine) [poly(GR)]. In some embodiments, the RAN protein is poly(Glycine-Aspartate) [poly(GD)]. In some embodiments, the RAN protein is poly(Glycine-Glutamate) [poly(GE)]. In some embodiments, the RAN protein is poly(Glycine-Glutamine) [poly(GQ)]. In some embodiments, the RAN protein is poly(Glycine-Leucine) [poly(GL)]. In some embodiments, the RAN protein is poly(Glycine-Lysine) [poly(GK)]. In some embodiments, the RAN protein is poly(Glycine-Proline) [(poly(GP)]. In some embodiments, the RAN protein is poly(Glycine-Threonine) [poly(GT)]. In some embodiments, the RAN protein is poly(Leucine) [polyLeu or poly(L)]. In some embodiments, the RAN protein is poly(Leucine-Proline) [poly(LP)]. In some embodiments, the RAN protein is poly(Leucine-Serine) [poly(LS)]. In some embodiments, the RAN protein is poly(phenylalanine-proline) [poly(FP)]. In some embodiments, the RAN protein is poly(Proline) [poly(P)]. In some embodiments, the RAN protein is poly(Proline-Alanine) [poly(PA)]. In some embodiments, the RAN protein is poly(Proline-Arginine) [poly(PR)]. In some embodiments, the RAN protein is poly(Serine) [polySer]. In some embodiments, the RAN protein is poly(Serine-Proline) [poly(SP)]. In some embodiments, the RAN protein is poly(Tryptophan-Alanine) [poly(WA)]. In some embodiments, the RAN protein is poly(Valine-Proline) [poly(VP)]. In some embodiments, the RAN protein is poly(Leucine-Proline-Alanine-Cysteine) [poly(LPAC)]. In some embodiments, the RAN protein is poly(Glutamine-Alanine-Glycine-Arginine) [poly(QAGR)]. In some embodiments, the RAN protein is Poly(Isoleucine-Leucine-Phenylalanine-Tyrosine-Serine) [poly(ILFTS)]. In some embodiments, the RAN protein is Poly(Phenylalanine-Histidine-Serine-Isoleucine-Proline) [poly(FHSIP)]. In some embodiments, the RAN protein is Poly(Tryptophan-Asparagine-Glycine-Methionine-Glutamine) [poly(WNGMQ)]. In some embodiments, the RAN protein is poly(PGGRGE). In some embodiments, the RAN protein is poly(FTPLSLPV). In some embodiments, the RAN protein is poly(LLPSPSRC). In some embodiments, the RAN protein is poly(YSPLPPGV). In some embodiments, the RAN protein is poly(HREGEGSK). In some embodiments, the RAN protein is poly(TGRERGVN). In some embodiments, the RAN protein is poly(GRQRGVNT). In some embodiments, the RAN protein is poly(GSKHREAE).

In certain embodiments, the method comprises reducing the level of RAN proteins that are poly-(Glycine-Leucine), poly-(Tryptophan-Alanine), poly-(Glutamine-Alanine), poly-(Glycine-Proline), and/or poly-(Proline-Arginine).

In certain embodiments, the method comprises reducing the level of RAN proteins that are poly-(Glutamine), which are associated with spinocerebellar ataxia type 12.

In certain embodiments, the method comprises reducing the level of RAN proteins that are poly-(Alanine), poly-(Leucine), poly-(Serine), and/or poly-(Cysteine), which are associated with DM1; spinocerebellar ataxia types 1, 2, 3, 6, 7, 8, 12, 17; spinal bulbar muscular atrophy; dentatorubral-pallidoluysian atrophy (DRPLA); and Huntington's disease.

In certain embodiments, the method comprises reducing the level of RAN proteins that are poly-(Glutamine), poly-(Alanine), poly-(Leucine), poly-(Serine), and/or poly-(Cysteine), which are associated with Huntington's disease-like 2 syndrome (HDL2); and Fuch's endothelial corneal dystrophy (FECD).

In certain embodiments, the method comprises reducing the level of RAN proteins that are poly-(Arginine), poly-(Glycine), poly-(Alanine), and/or poly-(Proline), which are associated with Fragile X syndrome (FXS); FRAXA; disorders related to 7p11.2 folate-sensitive fragile site FRA7A; disorders related to folate-sensitive fragile site 2q11 FRA2A; and Fragile XE syndrome (FRAXE).

In certain embodiments, the method comprises reducing the level of RAN proteins that are poly-(Alanine), poly-(Leucine), poly-(Serine), poly-(Cysteine), or poly-(Leu-Pro-Ala-Cys), which are associated with DM2.

In certain embodiments, the method comprises reducing the level of RAN proteins that are poly-(Gln-Ala-Gly-Arg), which are associated with DM2.

In certain embodiments, the method comprises reducing the level of RAN proteins that are poly-(Gly-Pro), poly-(Gly-Arg), poly-(Gly-Ala), poly-(Pro-Ala), or poly-(Pro-Arg), which are associated with sense C9ORFf72 ALS and C9ORF72 FTD.

In certain embodiments, the method comprises reducing the level of RAN proteins that are poly-(Pro-Ala), poly-(Pro-Arg), poly-(Gly-Pro), poly-(Pro-Ala), or poly-(Pro-Arg), which are associated with antisense C9ORFf72 ALS and antisense C9ORF72 FTD.

In certain embodiments, the method comprises reducing the level of RAN proteins that are poly-(Tryptophan-Asparagine-Glycine-Methionine-Glutamine) or poly-(Phenylalanine-Histidine-Serine-Isoleucine-Proline), which are associated with spinocerebellar ataxia type 31.

In certain embodiments, the method comprises reducing the level of RAN proteins that are poly-(Isoleucine-Leucine-Phenylalanine-Tyrosine-Serine), which are associated with spinocerebellar ataxia type 10.

In certain embodiments, the number of poly-amino acid repeats in the RAN protein is at least 25. In certain embodiments, the number of poly-amino acid repeats in the RAN protein is at least 35. In certain embodiments, the number of poly-amino acid repeats in the RAN protein is at least 45. In certain embodiments, the number of poly-amino acid repeats in the RAN protein is at least 50. In certain embodiments, the number of poly-amino acid repeats in the RAN protein is at least 70. In certain embodiments, the number of poly-amino acid repeats in the RAN protein is at least 80. In certain embodiments, the number of poly-amino acid repeats in the RAN protein is at least 90. In certain embodiments, the number of poly-amino acid repeats in the RAN protein is at least 100. In certain embodiments, the number of poly-amino acid repeats in the RAN protein is at least 120. In certain embodiments, the number of poly-amino acid repeats in the RAN protein is at least 150. In certain embodiments, the number of poly-amino acid repeats in the RAN protein is at least 200. In certain embodiments, the number of poly-amino acid repeats in the RAN protein is at least 250.

In certain embodiments, the neurological disease to be treated is associated with repeat expansions (e.g., repeat expansion mutations that undergo RAN protein translation). In certain embodiments, the neurological disease is associated with the expression of RAN proteins. In some embodiments, the repeat expansions comprise one or more of comprise AGGAT expansions, ATCCT expansions, ATCCT expansions, CAG expansions, CAGG expansions, CCAAGA expansions, CCAAGG expansions, CCACGA expansions, CCACGC expansions, CCACGG expansions, CCACGT expansions, CCCAGA expansions, CCCAGG expansions, CCCCGA expansions, CCCCGC expansions, CCCCGG expansions, CCCCGT expansions, CCGAGA expansions, CCGAGG expansions, CCGCGA expansions, CCGCGC expansions, CCGCGG expansions, CCGCGT expansions, CCTAGA expansions, CCTAGG expansions, CCTCGA expansions, CCTCGC expansions, CCTCGG expansions, CCTCGT expansions, CCTG expansions, CTG expansions, GAGAGG expansions, GGCCCA expansions, GGCCCC expansions, GGCCTG expansions, GGGGCA expansions, GGGGCC expansions, TGGAA expansions, and/or TGGGCC expansions.

In some embodiments, the repeat expansions comprise AGGAT expansions. In some embodiments, the repeat expansions comprise ATCCT expansions. In some embodiments, the repeat expansions comprise ATCCT expansions. In some embodiments, the repeat expansions comprise CAG expansions. In some embodiments, the repeat expansions comprise CAGG expansions. In some embodiments, the repeat expansions comprise CCAAGA expansions. In some embodiments, the repeat expansions comprise CCAAGG expansions. In some embodiments, the repeat expansions comprise CCACGA expansions. In some embodiments, the repeat expansions comprise CCACGC expansions. In some embodiments, the repeat expansions comprise CCACGG expansions. In some embodiments, the repeat expansions comprise CCACGT expansions. In some embodiments, the repeat expansions comprise CCCAGA expansions. In some embodiments, the repeat expansions comprise CCCAGG expansions. In some embodiments, the repeat expansions comprise CCCCGA expansions. In some embodiments, the repeat expansions comprise CCCCGC expansions. In some embodiments, the repeat expansions comprise CCCCGG expansions. In some embodiments, the repeat expansions comprise CCCCGT expansions. In some embodiments, the repeat expansions comprise CCGAGA expansions. In some embodiments, the repeat expansions comprise CCGAGG expansions. In some embodiments, the repeat expansions comprise CCGCGA expansions. In some embodiments, the repeat expansions comprise CCGCGC expansions. In some embodiments, the repeat expansions comprise CCGCGG expansions. In some embodiments, the repeat expansions comprise CCGCGT expansions. In some embodiments, the repeat expansions comprise CCTAGA expansions. In some embodiments, the repeat expansions comprise CCTAGG expansions. In some embodiments, the repeat expansions comprise CCTCGA expansions. In some embodiments, the repeat expansions comprise CCTCGC expansions. In some embodiments, the repeat expansions comprise CCTCGG expansions. In some embodiments, the repeat expansions comprise CCTCGT expansions. In some embodiments, the repeat expansions comprise CCTG expansions. In some embodiments, the repeat expansions comprise CTG expansions. In some embodiments, the repeat expansions comprise GAGAGG expansions. In some embodiments, the repeat expansions comprise GGCCCA expansions. In some embodiments, the repeat expansions comprise GGCCCC expansions. In some embodiments, the repeat expansions comprise GGCCTG expansions. In some embodiments, the repeat expansions comprise GGGGCA expansions. In some embodiments, the repeat expansions comprise GGGGCC expansions. In some embodiments, the repeat expansions comprise TGGAA expansions. In some embodiments, the repeat expansions comprise TGGGCC expansions. In some embodiments, the repeat expansions comprise GGGGCC expansions and GGCCCC expansions. In some embodiments, the repeat expansions comprise CAG expansions and CTG expansions. In some embodiments, the repeat expansions comprise CAGG expansions and CCTG expansions. In some embodiments, the repeat expansions comprise TGGAA expansions. In some embodiments, the repeat expansions comprise GGCCTG expansions, TGGGCC expansions, or GGCCCA expansions. In some embodiments, the repeat expansions comprise ATCCT expansions, ATCCT expansions, or AGGAT expansions.

In some embodiments, the neurological disease is amyotrophic lateral sclerosis (ALS) (e.g., C9ORFf72 ALS), frontotemporal dementia (FTD) (e.g., C9ORF72 FTD), spinocerebellar ataxia (e.g., spinocerebellar ataxia type 1, spinocerebellar ataxia type 2, spinocerebellar ataxia type 3, spinocerebellar ataxia type 8, spinocerebellar ataxia type 6, spinocerebellar ataxia type 7, spinocerebellar ataxia type 10, spinocerebellar ataxia type 12, spinocerebellar ataxia type 17, spinocerebellar ataxia type 31, or spinocerebellar ataxia type 36), myotonic dystrophy type 1, myotonic dystrophy type 2, Fuch's corneal endothelial dystrophy, spinal bulbar muscular atrophy, dentatorubral-pallidoluysian atrophy, Huntington's disease, Alzheimer's disease, Fragile X Tremor Ataxia Syndrome (FXTAS), Huntington's disease-like 2 syndrome (HDL2), Fragile X syndrome (FXS), disorders related to 7p11.2 folate-sensitive fragile site FRA7A, disorders related to folate-sensitive fragile site 2q11 FRA2A, or Fragile XE syndrome (FRAXE). In some embodiments, the neurological disease is amyotrophic lateral sclerosis (ALS). In some embodiments, the neurological disease is C9ORFf72 ALS. In some embodiments, the neurological disease is frontotemporal dementia (FTD). In some embodiments, the neurological disease is C9ORF72 FTD. In some embodiments, the neurological disease is spinocerebellar ataxia. In some embodiments, the neurological disease is spinocerebellar ataxia type 1. In some embodiments, the neurological disease is spinocerebellar ataxia type 2. In some embodiments, the neurological disease is spinocerebellar ataxia type 3. In some embodiments, the neurological disease is spinocerebellar ataxia type 8. In some embodiments, the neurological disease is spinocerebellar ataxia type 6. In some embodiments, the neurological disease is spinocerebellar ataxia type 7. In some embodiments, the neurological disease is spinocerebellar ataxia type 10. In some embodiments, the neurological disease is spinocerebellar ataxia type 12. In some embodiments, the neurological disease is spinocerebellar ataxia type 17. In some embodiments, the neurological disease is spinocerebellar ataxia type 31. In some embodiments, the neurological disease is or spinocerebellar ataxia type 36. In some embodiments, the neurological disease is myotonic dystrophy type 1. In some embodiments, the neurological disease is myotonic dystrophy type 2. In some embodiments, the neurological disease is Fuch's corneal endothelial dystrophy. In some embodiments, the neurological disease is spinal bulbar muscular atrophy. In some embodiments, the neurological disease is dentatorubral-pallidoluysian atrophy. In some embodiments, the neurological disease is Huntington's disease. In some embodiments, the neurological disease is Alzheimer's disease. In some embodiments, the neurological disease is Fragile X Tremor Ataxia Syndrome (FXTAS). In some embodiments, the neurological disease is Huntington's disease-like 2 syndrome (HDL2). In some embodiments, the neurological disease is Fragile X syndrome (FXS). In some embodiments, the neurological disease is disorders related to 7p11.2 folate-sensitive fragile site FRA7A. In some embodiments, the neurological disease is disorders related to folate-sensitive fragile site 2q11 FRA2A. In some embodiments, the neurological disease is or Fragile XE syndrome (FRAXE). In some embodiments, the neurological disease is amyotrophic lateral sclerosis (ALS) or frontotemporal dementia (FTD). In some embodiments, the neurological disease is C9ORFf72 ALS or C9ORFf72 FTD. In some embodiments, the neurological disease is Alzheimer's disease. In some embodiments, the neurological disease is spinocerebellar ataxia. In some embodiments, the neurological disease is spinocerebellar ataxia type 1, spinocerebellar ataxia type 2, spinocerebellar ataxia type 3, or spinocerebellar ataxia type 8. In some embodiments, the neurological disease is spinocerebellar ataxia type 6, spinocerebellar ataxia type 7, spinocerebellar ataxia type 10, spinocerebellar ataxia type 12, spinocerebellar ataxia type 17, spinocerebellar ataxia type 31, or spinocerebellar ataxia type 36. In some embodiments, the neurological disease is myotonic dystrophy type 1, myotonic dystrophy type 2, or Fuch's corneal endothelial dystrophy. In some embodiments, the neurological disease is spinal bulbar muscular atrophy or dentatorubral-pallidoluysian atrophy. In some embodiments, the neurological disease is Huntington's disease. In some embodiments, the neurological disease is Fragile X Tremor Ataxia Syndrome (FXTAS). In some embodiments, the neurological disease is Huntington's disease-like 2 syndrome (HDL2); Fragile X syndrome (FXS); disorders related to 7p11.2 folate-sensitive fragile site FRA7A; disorders related to folate-sensitive fragile site 2q11 FRA2A; or Fragile XE syndrome (FRAXE).

In some embodiments, the neurological disease is a neurodegenerative disorder.

In certain embodiments, the neurological disease being treated is a neurodegenerative disorder. In certain embodiments, the neurological disease being treated is a neuromuscular disorder. In certain embodiments, the neurological disease is associated with GGGGCC expansions and/or GGCCCC expansions. In certain embodiments, the neurological disease is associated with GGGGCC expansions and GGCCCC expansions. In certain embodiments, the neurological disease is associated with GGGGCC expansions. In certain embodiments, the neurological disease is associated with GGCCCC expansions. In certain embodiments, the neurological disease is associated with GGGGCC expansions and/or GGCCCC expansions is amyotrophic lateral sclerosis (ALS). In certain embodiments, the neurological disease is associated with GGGGCC expansions and/or GGCCCC expansions is frontotemporal dementia (FTD). In certain embodiments, the neurological disease is associated with GGGGCC expansions and/or GGCCCC expansions is C9ORFf72 ALS. In certain embodiments, the neurological disease is associated with GGGGCC expansions and/or GGCCCC expansions C9ORF72 FTD.

In certain embodiments, the neurological disease is associated with CAG expansions and/or CTG expansions. In certain embodiments, the neurological disease is associated with CAG expansions and CTG expansions. In certain embodiments, the neurological disease is associated with CAG expansions. In certain embodiments, the neurological disease is associated with CTG expansions. In certain embodiments, the neurological disease is associated with CAG expansions and/or CTG expansions is spinocerebellar ataxia (SCA). In certain embodiments, the neurological disease is associated with TGGAA expansions. In certain embodiments, the neurological disease is associated with TGGAA expansions is spinocerebellar ataxia. In certain embodiments, the neurological disease is associated with TGGAA expansions is spinocerebellar ataxia type 31. In certain embodiments, the neurological disease is associated with GGCCTG expansions. In certain embodiments, the neurological disease is associated with GGCCTG expansions is spinocerebellar ataxia type 36. In certain embodiments, the neurological disease is associated with TGGGCC expansions. In certain embodiments, the neurological disease is associated with 5′ TGGGCC expansions. In certain embodiments, the neurological disease is associated with TGGGCC expansions is spinocerebellar ataxia type 36. In certain embodiments, the neurological disease is associated with 5′ TGGGCC expansions is spinocerebellar ataxia type 36. In certain embodiments, the neurological disease is associated with GGCCCA expansions of another DNA strand. In certain embodiments, the neurological disease is associated with 5′ GGCCCA expansions of another DNA strand. In certain embodiments, the neurological disease is associated with 5′ GGCCCA expansions of another DNA strand is spinocerebellar ataxia type 36. In certain embodiments, the neurological disease is associated with ATCCT expansions. In certain embodiments, the neurological disease is associated with 5′ ATCCT expansions. In certain embodiments, the neurological disease is associated with ATCCT expansions is spinocerebellar ataxia type 10. In certain embodiments, the neurological disease is associated with 5′ ATCCT expansions is spinocerebellar ataxia type 10. In certain embodiments, the neurological disease is associated with AGGAT expansions of another DNA strand. In certain embodiments, the neurological disease is associated with 5′ AGGAT expansions of another DNA strand. In certain embodiments, the neurological disease is associated with AGGAT expansions of another DNA strand is spinocerebellar ataxia type 10.

In certain embodiments, the neurological disease is associated with 5′ AGGAT expansions of another DNA strand is spinocerebellar ataxia type 10.

In embodiments, the neurological disease is associated with CAGG expansions and/or CCTG expansions. In embodiments, the neurological disease is associated with CAGG expansions and CCTG expansions. In embodiments, the neurological disease is associated with CAGG expansions. In embodiments, the neurological disease is associated with CCTG expansions. In certain embodiments, the neurological disease is associated with CAGG expansions and/or CCTG expansions is myotonic dystrophy type 2. In certain embodiments, the neurological disease is associated with RAN protein accumulation.

In certain embodiments, the neurological disease is a neurodegenerative disorder, and is associated with a RAN protein where the number of poly-amino acid repeats in the RAN protein is at least 35. In certain embodiments, the neurological disease is a neurodegenerative disorder, and is associated with a RAN protein where the number of poly-amino acid repeats in the RAN protein is at least 50. In certain embodiments, the neurological disease is a neurodegenerative disorder, and is associated with a RAN protein where the number of poly-amino acid repeats in the RAN protein is at least 70. In certain embodiments, the neurological disease is spinal bulbar muscular atrophy or dentatorubral-pallidoluysian atrophy. In certain embodiments, the neurological disease is spinal bulbar muscular atrophy. In certain embodiments, the neurological disease is dentatorubral-pallidoluysian atrophy.

In some embodiments, the ratio of the compound of Formula (I) to the second compound of Formula (I) administered is about 1:1, about 1:2, about 1:2.5, about 1:4, about 1:5, about 1:8, about 1:16, about 16:1, about 8:1, about 5:1, about 4:1, about 1:2.5, or about 2:1. In some embodiments, the ratio of the compound of Formula (I) to the second compound of Formula (I) administered is about 1:2, about 1:4, about 1:8, or about 1:16. In some embodiments, the ratio of the compound of Formula (I) to the second compound of Formula (I) administered is about 1:18 or about 1:16.

In some embodiments, the ratio of the compound of Formula (I) to the compound of Formula (II) administered is about 1:1, about 1:2, about 1:2.5, about 1:4, about 1:5, about 1:8, about 1:16, about 16:1, about 8:1, about 5:1, about 4:1, about 1:2.5, or about 2:1. In some embodiments the ratio of the compound of Formula (I) to the compound of Formula (II) administered is about 1:2, about 1:4, about 1:8, or about 1:16. In some embodiments, the ratio of the compound of Formula (I) to the compound of Formula (II) administered is about 1:18 or about 1:16.

In some embodiments, a method as provided herein further comprises administering an additional therapeutic agent for treating a neurological disease.

In certain embodiments, a method as provided herein further comprises administering a third compound selected from the group consisting of a compound of Formula (I), or a pharmaceutically acceptable salt, co-crystal, tautomer, stereoisomer, solvate, hydrate, polymorph, isotopically enriched derivative, or prodrug thereof, and a compound of Formula (II), or a pharmaceutically acceptable salt, co-crystal, tautomer, stereoisomer, solvate, hydrate, polymorph, isotopically enriched derivative, or prodrug thereof.

In certain embodiments, the compound of Formula (I) and the second compound of Formula (I) are administered in separate pharmaceutical compositions. In some embodiments, the compound of Formula (I) and the second compound of Formula (I) are administered concurrently. In certain embodiments, the compound of Formula (I) and the second compound of Formula (I) are administered sequentially. In some embodiments, the compound of Formula (I) is administered before the second compound of Formula (I). In certain embodiments, the second compound of Formula (I) is administered before the compound of Formula (I).

In some embodiments, the administration of the combination of compounds exhibits synergy compared to administration of the compound of Formula (I) or the second compound of Formula (I) alone.

In certain embodiments, a lower effective dose of the compound of Formula (I) and/or the second compound of Formula (I) may be administered as compared to when said compounds are administered alone.

In some embodiments, the compound of Formula (I) and the compound of Formula (II) are administered in separate pharmaceutical compositions. In certain embodiments, the compound of Formula (I) and the compound of Formula (II) are administered concurrently. In some embodiments, the compound of Formula (I) and the compound of Formula (II) are administered sequentially. In certain embodiments, the compound of Formula (I) is administered before the compound of Formula (II). In some embodiments, the compound of Formula (II) is administered before the compound of Formula (I).

In certain embodiments, the administration of the combination of compounds exhibits synergy compared to administration of the compound of Formula (I) or the compound of Formula (II) alone.

In certain embodiments, a lower effective dose of the compound of Formula (I) and/or the compound of Formula (II) may be administered as compared to when said compounds are administered alone.

### Pharmaceutical Compositions

In some embodiments, provided herein are pharmaceutical compositions comprising: (a) a compound of Formula (I):

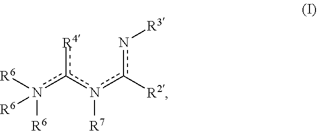

or a pharmaceutically acceptable salt, co-crystal, tautomer, stereoisomer, solvate, hydrate, polymorph, isotopically enriched derivative, or prodrug thereof, wherein [symbol] , R^{2′}, R³, R^{4′}, R⁶, and R⁷ are defined herein; and (b) a second compound of Formula (I), or a pharmaceutically acceptable salt, co-crystal, tautomer, stereoisomer, solvate, hydrate, polymorph, isotopically enriched derivative, or prodrug thereof.

In certain embodiments, provided herein are pharmaceutical compositions comprising: (a) a compound of Formula (I):

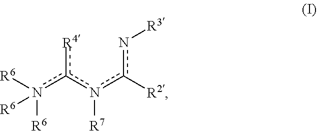

or a pharmaceutically acceptable salt, co-crystal, tautomer, stereoisomer, solvate, hydrate, polymorph, isotopically enriched derivative, or prodrug thereof, wherein [symbol] , R^{2′}, R³, R^{4′}, R⁶, and R⁷ are defined herein; and (b) a compound of Formula (II):

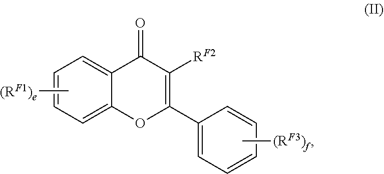

or a pharmaceutically acceptable salt, co-crystal, tautomer, stereoisomer, solvate, hydrate, polymorph, isotopically enriched derivative, or prodrug thereof, wherein R^{F1}, R^{F2}, R^{F3}, e, and f are as defined herein.

In certain embodiments, provided herein are pharmaceutical compositions comprise a compound of Formula (II):

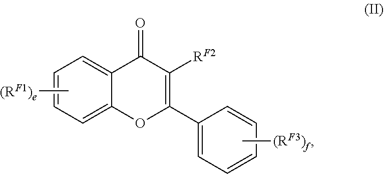

or a pharmaceutically acceptable salt, co-crystal, tautomer, stereoisomer, solvate, hydrate, polymorph, isotopically enriched derivative, or prodrug thereof, wherein R^{F1}, R^{F2}, R^{F3}, e, and f are as defined herein.

In some embodiments, the compositions provided herein comprise (a) a compound of Formula (I):

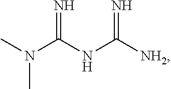

or a pharmaceutically acceptable salt, co-crystal, tautomer, stereoisomer, solvate, hydrate, polymorph, isotopically enriched derivative, or prodrug thereof; and (b) a compound of Formula (II):

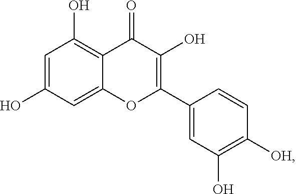 [SMILES: O=c1c(O)c(-c2ccc(O)c(O)c2)oc2cc(O)cc(O)c12]

or a pharmaceutically acceptable salt, co-crystal, tautomer, stereoisomer, solvate, hydrate, polymorph, isotopically enriched derivative, or prodrug thereof. In some embodiments, the compositions provided herein comprise: (a) a compound of Formula (I):

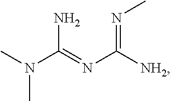

or a pharmaceutically acceptable salt, co-crystal, tautomer, stereoisomer, solvate, hydrate, polymorph, isotopically enriched derivative, or prodrug thereof; and (b) a compound of Formula (II):

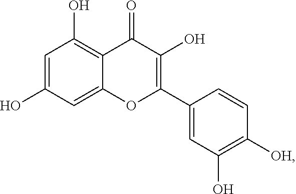 [SMILES: O=c1c(O)c(-c2ccc(O)c(O)c2)oc2cc(O)cc(O)c12]

or a pharmaceutically acceptable salt, co-crystal, tautomer, stereoisomer, solvate, hydrate, polymorph, isotopically enriched derivative, or prodrug thereof. In certain embodiments, the compositions provided herein comprise: (a) a compound of Formula (I):

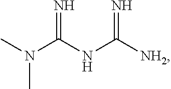

or a pharmaceutically acceptable salt, co-crystal, tautomer, stereoisomer, solvate, hydrate, polymorph, isotopically enriched derivative, or prodrug thereof; and (b) a compound of Formula (II):

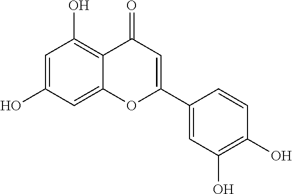 [SMILES: O=c1cc(-c2ccc(O)c(O)c2)oc2cc(O)cc(O)c12]

or a pharmaceutically acceptable salt, co-crystal, tautomer, stereoisomer, solvate, hydrate, polymorph, isotopically enriched derivative, or prodrug thereof. In some embodiments, the compositions provided herein comprise (a) a compound of Formula (I):

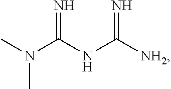

or a pharmaceutically acceptable salt, co-crystal, tautomer, stereoisomer, solvate, hydrate, polymorph, isotopically enriched derivative, or prodrug thereof; and (b) a second compound of Formula (I):

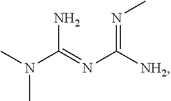

or a pharmaceutically acceptable salt, co-crystal, tautomer, stereoisomer, solvate, hydrate, polymorph, isotopically enriched derivative, or prodrug thereof.

The present disclosure also provides pharmaceutical compositions comprising a compound of Formula (I), a second compound of Formula (I), and a pharmaceutically acceptable excipient. In certain embodiments, the pharmaceutical composition comprises a compound of Formula (I), a compound of Formula (II), and a pharmaceutically acceptable excipient.

In certain embodiments, a composition as provided herein further comprises a third compound selected from the group consisting of a compound of Formula (I), or a pharmaceutically acceptable salt, co-crystal, tautomer, stereoisomer, solvate, hydrate, polymorph, isotopically enriched derivative, or prodrug thereof, and a compound of Formula (II), or a pharmaceutically acceptable salt, co-crystal, tautomer, stereoisomer, solvate, hydrate, polymorph, isotopically enriched derivative, or prodrug thereof.

In certain embodiments, a compound of Formula (I) and a compound of Formula (II) or a second compound of Formula (I) is provided in an effective amount in a pharmaceutical composition. In certain embodiments, the effective amount is a therapeutically effective amount. In certain embodiments, the effective amount is a prophylactically effective amount. In certain embodiments, a therapeutically effective amount is an amount effective in reducing repeat expansions. In certain embodiments, a therapeutically effective amount is an amount effective in reducing the transcription of RNAs that produce RAN proteins. In certain embodiments, a therapeutically effective amount is an amount effective in reducing the translation of RAN proteins. In certain embodiments, a therapeutically effective amount is an amount effective in reducing the level of one or more RAN proteins in a subject. In certain embodiments, a therapeutically effective amount is an amount effective for treating a neurological disease associated with repeat expansions. In certain embodiments, a therapeutically effective amount is an amount effective in reducing the level of one or more RAN proteins and treating a neurological disease associated with repeat expansions. In certain embodiments, a therapeutically effective amount is an amount effective in reducing the level of one or more RAN proteins and treating a neurological disease associated with RAN protein accumulation. In certain embodiments, a therapeutically effective amount is an amount effective in reducing the accumulation of RAN proteins.

In certain embodiments, the effective amount is an amount effective in reducing the level of one or more RAN proteins by at least 10%, at least 20%, at least 30%, at least 40%, at least 50%, at least 60%, at least 70%, at least 80%, at least 90%, at least 95%, or at least 98%. In certain embodiments, the effective amount is an amount effective in reducing the translation of RAN proteins by at least 10%, at least 20%, at least 30%, at least 40%, at least 50%, at least 60%, at least 70%, at least 80%, at least 90%, at least 95%, or at least 98%.

Pharmaceutical compositions described herein can be prepared by any method known in the art of pharmacology. In general, such preparatory methods include bringing the compounds described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol*), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum©), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof. The exemplary liquid dosage forms in certain embodiments are formulated for ease of swallowing, or for administration via feeding tube.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

In some embodiments, the ratio of the compound of Formula (I) to the second compound of Formula (I) in the composition is about 1:1, about 1:2, about 1:2.5, about 1:4, about 1:5, about 1:8, about 1:16, about 16:1, about 8:1, about 5:1, about 4:1, about 1:2.5, or about 2:1. In some embodiments, the ratio of the compound of Formula (I) to the second compound of Formula (I) in the composition is about 1:2, about 1:4, about 1:8, or about 1:16. In some embodiments, the ratio of the compound of Formula (I) to the second compound of Formula (I) is about 1:18 or about 1:16.

In some embodiments, the ratio of the compound of Formula (I) to the compound of Formula (II) in the composition is about 1:1, about 1:2, about 1:2.5, about 1:4, about 1:5, about 1:8, about 1:16, about 16:1, about 8:1, about 5:1, about 4:1, about 1:2.5, or about 2:1. In some embodiments the ratio of the compound of Formula (I) to the compound of Formula (II) in the composition is about 1:2, about 1:4, about 1:8, or about 1:16. In some embodiments, the ratio of the compound of Formula (I) to the compound of Formula (II) is about 1:18 or about 1:16.

### Administration

The compounds used in the methods provided herein and/or the compositions provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific compounds/composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

A compounds used in the methods provided herein and/or the compositions provided herein are typically administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compounds or pharmaceutical composition described herein is suitable for topical administration to the eye of a subject.

The exact amount of the compounds utilized herein and/or composition provided herein required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of a compound described herein. In certain embodiments, when multiple doses are administered to a subject or applied to a biological sample, tissue, or cell, the frequency of administering the multiple doses to the subject or applying the multiple doses to the biological sample, tissue, or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the biological sample, tissue, or cell is one dose per day. In certain embodiments, the frequency of administering the multiple doses to the subject or applying the multiple doses to the biological sample, tissue, or cell is two doses per day. In certain embodiments, the frequency of administering the multiple doses to the subject or applying the multiple doses to the biological sample, tissue, or cell is three doses per day. In certain embodiments, when multiple doses are administered to a subject or applied to a biological sample, tissue, or cell, the duration between the first dose and last dose of the multiple doses is one day, two days, four days, one week, two weeks, three weeks, one month, two months, three months, four months, six months, eight months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

In certain embodiments, a compound described herein is formulated as a tablet with hydrobromic acid. In certain embodiments, a compound described herein is formulated as a tablet with phosphoric acid. In certain embodiments, a compound described herein is formulated as a tablet with sulfuric acid. In certain embodiments, a compound described herein is formulated as a tablet with perchloric acid. In certain embodiments, a compound described herein is formulated as a tablet with an organic acid such as acetic acid, oxalic acid, maleic acid, tartaric acid, citric acid, succinic acid, or malonic acid. In certain embodiments, a compound described herein is formulated as a tablet using other methods known in the art such as ion exchange.

In certain embodiments, a compound of Formula (I) is formulated as a tablet with hydrochloride. In certain embodiments, a compound of Formula (I) is formulated as a tablet with a pharmaceutically acceptable salt derived from a suitable inorganic acid, organic acid, or organic base. In certain embodiments, a compound of Formula (I) is formulated as a tablet with HBr. In certain embodiments, a compound of Formula (I) is formulated as a metformin hydrochloride extended release tablet. In certain embodiments, a compound of Formula (I) is formulated as a metformin succinate or metformin fumarate salt.

In some embodiments, the methods provided herein comprise administering to the subject a dose of 200-1000 mg per day of the compound of Formula (I), or a pharmaceutically acceptable salt, co-crystal, tautomer, stereoisomer, solvate, hydrate, polymorph, isotopically enriched derivative, or prodrug thereof, and/or the second compound of Formula (I), or a pharmaceutically acceptable salt, co-crystal, tautomer, stereoisomer, solvate, hydrate, polymorph, isotopically enriched derivative, or prodrug thereof. In certain embodiments, a compound of Formula (I) is administered in doses of 500 mg twice a day or doses of 850 mg once a day. In certain embodiments, a compound of Formula (I) is administered in doses of at least 825 mg three times a day. In certain embodiments, a compound of Formula (I) is administered in doses of 825 mg. In certain embodiments, a compound of Formula (I) is administered in doses of 500 mg once a day. In certain embodiments, a compound of Formula (I) is administered in doses of 1000 mg once a day.

In some embodiments, a compound of Formula (I) is administered over a period of at least 10 days, at least 30 days, at least six months, or at least one year. In certain embodiments, the methods provided herein comprises administering to the subject a compound of Formula (I) over a period between 10 days to 30 days. In certain embodiments, the duration between the first dose and last dose of the multiple doses of a compound of Formula (I) is 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days. In certain embodiments, the duration between the first dose and last dose of the multiple doses of a compound of Formula (I) is at least the following number of days: 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days. In certain embodiments, the duration between the first dose and last dose of the multiple doses of metformin is 1 month, 2 months, 3 months, 4 months, 5 months, 6 months, multiple months, at least one year, multiple years, at least one decade, or multiple decades. In certain embodiments, the doses of a compound of Formula (I) are administered indefinitely. In certain embodiments, the doses of metformin are administered over a lifetime of the subject. In certain embodiments, a dose described herein is at least 500 mg, 600 mg, 650 mg, 750 mg, 700 mg, 800 mg, 825 mg, 850 mg, 900 mg, 950 mg, 1000 mg, 1500 mg, 2000 mg, 2500 mg, 3000 mg, 3500 mg, 4000 mg, 5000 mg, 8000 mg, 9000 mg, or 10,000 mg of a compound of Formula (I). In certain embodiments, the duration between the first dose and last dose of the multiple doses of a compound of Formula (I) is based on the duration required to prevent the accumulation of RAN proteins in a subject. In certain embodiments, the duration between the first dose and last dose of the multiple doses of a compound of Formula (I) is based on the duration required to reduce the level of RAN proteins in a subject. In certain embodiments, the multiple doses of a compound of Formula (I) are administered as prophylactic treatment to reduce the level of RAN proteins in a subject. The prophylactic treatment is long-term, in certain embodiments. In certain embodiments, the multiple doses of a compound of Formula (I) are administered as long-term therapeutic treatment to reduce the level of RAN proteins in a subject.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

The compound combinations or compositions described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, improve bioavailability, improve safety, reduce drug resistance, reduce and/or modify metabolism, inhibit excretion, and/or modify distribution in a subject, biological sample, tissue, or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both.

The compound combinations or compositions disclosed herein can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., neurological disease). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, and a combination thereof. In some embodiments, additional pharmaceutical agents include, but are not limited to, cardiovascular agents, anti-diabetic agents, and agents for treating and/or preventing a neurological disease. The additional pharmaceutical agents include, but are not limited to, anti-inflammatory agents or compounds (e.g., turmeric).

### Kits

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

In one aspect, provided herein are kits for treating a neurological disease associated with repeat expansions in a subject in need thereof comprising: a compound of Formula (I), or a pharmaceutically acceptable salt, co-crystal, tautomer, stereoisomer, solvate, hydrate, polymorph, isotopically enriched derivative, or prodrug thereof; a compound of Formula (I) or (II), or a pharmaceutically acceptable salt, co-crystal, tautomer, stereoisomer, solvate, hydrate, polymorph, isotopically enriched derivative, or prodrug thereof; and instructions for administering the compound of Formula (I), or the pharmaceutically acceptable salt, co-crystal, tautomer, stereoisomer, solvate, hydrate, polymorph, isotopically enriched derivative, or prodrug thereof, and the compound of Formula (I) or (II), or the pharmaceutically acceptable salt, co-crystal, tautomer, stereoisomer, solvate, hydrate, polymorph, isotopically enriched derivative, or prodrug thereof.

In another aspect, provided herein are kits for treating a neurological disease associated with repeat expansions in a subject in need thereof comprising: a compound of Formula (II), or a pharmaceutically acceptable salt, co-crystal, tautomer, stereoisomer, solvate, hydrate, polymorph, isotopically enriched derivative, or prodrug thereof; and instructions for administering the compound of Formula (II), or the pharmaceutically acceptable salt, co-crystal, tautomer, stereoisomer, solvate, hydrate, polymorph, isotopically enriched derivative, or prodrug thereof.

In another aspect, provided herein are kits for treating a neurological disease associated with repeat expansions in a subject in need thereof comprising: a composition as provided herein; and instructions for administering the composition.

Thus, in one aspect, provided herein are kits including one or more containers comprising one or more compounds of Formula (I), or a pharmaceutically acceptable salt, co-crystal, tautomer, stereoisomer, solvate, hydrate, polymorph, isotopically enriched derivative, or prodrug thereof, and/or a compound of Formula (II), or a pharmaceutically acceptable salt, co-crystal, tautomer, stereoisomer, solvate, hydrate, polymorph, isotopically enriched derivative, or prodrug thereof. In another aspect, provided herein are kits including a container comprising a composition as described herein. In certain embodiments, the kits are useful for treating a neurological disease) in a subject in need thereof. In certain embodiments, the kits are useful for preventing a neurological disease) in a subject in need thereof. In certain embodiments, the kits are useful for reducing the level of one or more RAN proteins (e.g., reducing the expression of RAN proteins) in a subject, biological sample, tissue, or cell. In certain embodiments, the kits are useful for reducing the accumulation of RAN proteins in a subject, biological sample, tissue, or cell. In certain embodiments, the kits are useful for modulating (e.g., reducing or inhibiting) RAN protein translation in a subject, biological sample, tissue, or cell.

In certain embodiments, a kit described herein further includes instructions for administering the compounds or compositions included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., a neurological disease) in a subject in need thereof. In certain embodiments, the kits and instructions provide for preventing a disease (e.g., a neurological disease) in a subject in need thereof. In certain embodiments, the kits and instructions provide for reducing the level of one or more RAN proteins in a subject, biological sample, tissue, or cell. In certain embodiments, the kits and instructions provide for reducing the accumulation of RAN proteins in a subject, biological sample, tissue, or cell. In certain embodiments, the kits and instructions provide for modulating (e.g., reducing or inhibiting) RAN protein translation in a subject, biological sample, tissue, or cell. A kit described herein may include one or more additional pharmaceutical agents described herein as a separate composition.

### EXAMPLES

In order that the present disclosure may be more fully understood, the following examples are set forth. The examples described in this application are offered to illustrate the compositions and methods provided herein and are not to be construed in any way as limiting their scope.

cDNA constructs. CAG construct (also known as A8*KMQ-3T) was previously described (Zu, T. et al. Non-ATG-initiated translation directed by microsatellite expansions. Proc Natl Acad Sci USA 108, 260-265, doi:10.1073/pnas.1013343108 (2011). These constructs contain six stop codons (two in each reading frame) upstream of the repeat expansion and three different reading frame specific C-terminal epitope tags.

Cell culture and treatment. HEK293T cells were cultured in DMEM medium supplemented with 10% fetal bovine serum and incubated at 37° C. in a humid atmosphere containing 5% CO₂. DNA transfections were performed using Lipofectamine 2000 Reagent (Thermo Fisher Scientific) according to the manufacturer's instructions. Forty-eight hours post-transfection, cells were collected and used for protein blotting. For compound 3 experiments, cells were treated with compound 3 (FCH Group) at 1, 2, or 0.5 mM for 24 hours, 24 hours after transfection with plasmid DNA. For Quercetin experiments, cells were treated with at 5, 10, 20, or 40 μM Qucercetin (Sigma) for 24 hours, 24 hours after transfection with plasmid DNA. For metformin treatments, cells were transfected with plasmids and simultaneously treated with at 2.5 or 5 mM metformin for 48 hours.

Antibodies. For western blotting the following primary antibodies were used: rabbit anti-Myc tag (Abcam, Cat. #ab9106, 1:200 dilution), mouse anti-HA tag (BioLegent, Cat. #901513), mouse anti-Flag tag (Sigma-Aldrich, Cat. #A8591, 1:1000 dilution), mouse anti-GAPDH (Millipore, Cat. #MAB374, 1:5000 dilution), rabbit anti-PKR (Abcam, Cat. #ab32506, 1:5000 dilution), rabbit anti-PKR (phospho T446) (Abcam, Cat. #ab32036, 1:1000 dilution), rabbit anti-PKR (phospho T451) (Abcam, Cat. #ab81303, 1:1000 dilution), rabbit anti-EIF2S1 (phospho S51) (Abcam, Cat. #ab32157, 1:1000 dilution), and mouse anti-eIF2a (Santa Cruz, sc-133227, 1:2000 dilution).

For western blotting the following secondary antibodies were used: sheep anti-mouse IgG HRP (GE Healthcare, Cat. #NA931, 1:2000 dilution), and donkey anti-rabbit IgG HRP (GE Healthcare, Cat. #NA934, 1:2000 dilution).

Western blotting. Transfected cells in each well of a six-well tissue-culture plate were rinsed with PBS and lysed in 300 μL RIPA buffer with protease and phosphatase inhibitor cocktails (Sigma-Aldrich) for 45 min on ice. DNA was sheared by repeated passage through a 21-gauge needle. The cell lysates were centrifuged at 16,000×g for 15 min at 4° C., and the supernatant was collected. The protein concentration of the cell lysate was determined using the protein assay dye reagent (Bio-Rad). Twenty micrograms of protein were separated in a 4-12% Criterion Bis-Tris gel (Bio-Rad) and transferred to a nitrocellulose membrane (GE Healthcare). The membrane was blocked in 5% dry milk in PBS containing 0.05% Tween-20 (PBS-T) and probed with primary antibody in blocking solution. After the membrane was incubated with an HRP-conjugated secondary antibody (GE Healthcare), bands were visualized by Western Lightning Plus-ECL (PerkinElmer) and X-ray film.

Quantification and statistical analysis. For densitometry, each experiment was done at least three independent times and results were quantified using ImageJ Software. Differences between groups were determined by one-way ANOVA with Tukey, Dunnett analyses for multiple comparisons, or Holm-Sidak analyses for multiple comparisons.

Gait analysis. Digital video images of the underside of the mouse were collected with a high-speed video camera from below the transparent belt of a motorized treadmill (DigiGait™ Imaging system, Mouse Specific). Female C9orf72 ALS/FTD mice (C9) mice and their littermates (non-transgenic mice (NT)) were tested at 18 weeks of age with treatment beginning at 8 weeks of age. Each mouse was allowed to explore the treadmill compartment with the motor speed set to 14 cm/s for 1 min, then the motor speed was increased to 24c m/s for video recording. Only video recordings in which the mouse walked straight ahead with a constant relative position with respect to the camera were used for analysis. Data from each hind paw was analyzed with DigiGait automated gait analysis software (Mouse Specifics). For additional details, see Neuron, 2016, 90(3), 521-34. doi: 10.1016/j.neuron.2016.04.005. Mock treatment is vehicle only, meaning chow without quercetin.

TABLE 1
DigiGait analysis of 18 week female mice

| | Quercetin-NT | Mock Treatment-NT |
|---|---|---|
| | vs | vs |
| | Quercetin C9 | Mock Treatment-C9 |
| Swing (%) | ns | p = 0.0034 |
| SwingStride (%) | ns | ns |
| Brake (s) | ns | ns |
| BrakeStride (%) | ns | p = 0.0062 |
| Propel (s) | ns | p = 0.0155 |
| PropelStride (%) | ns | ns |
| Stance (s) | ns | ns |
| StanceStride (%) | ns | ns |
| Stride (s) | ns | p = 0.0059 |
| BrakeStance (%) | ns | p = 0.0110 |
| PropelStance (%) | ns | p = 0.0110 |
| Stance/Swing (real#) | ns | ns |
| StrideLength (cm) | ns | p = 0.0039 |
| Stride Frequency (steps/s) | ns | p = 0.0061 |
| PawAngle (deg) | ns | p = 0.0219 |
| Absolute PawAngle (deg) | ns | ns |
| Paw Angle Variability (deg) | ns | ns |
| StanceWidth (cm) | ns | ns |
| StepAngle (deg) | ns | ns |
| SLVar (cm) | ns | ns |
| SWVar (cm) | ns | ns |
| StepAngle Var (deg) | ns | p = 0.0067 |
| Steps (real#) | ns | ns |
| Stride Length CV (CV %) | ns | ns |
| Stance Width CV (CV %) | ns | ns |
| Step Angle CV (CV %) | ns | p = 0.0042 |
| Swing Duration CV (CV %) | ns | ns |
| Paw Area at Peak Stance | ns | ns |
| in sq. cm (cm²) | | |
| Paw Area Variability at Peak | ns | p = 0.0477 |
| Stance in sq. cm (cm²) | | |
| Hind Limb Shared Stance Time (s) | ns | ns |
| Shared Stance (%) | ns | p = 0.0101 |
| StanceFactor (real#) | ns | ns |
| Gait Symmetry (real#) | ns | ns |
| MAX dA/dT (cm²/s) | ns | ns |
| MIN dA/dT (cm²/s) | ns | ns |
| Tau - Propulsion (real#) | ns | ns |
| Overlap Distance (cm) | ns | ns |
| PawPlacementPositioning[PPP] | ns | ns |
| (cm) | | |
| Ataxia Coefficient (real#) | ns | ns |
| Midline Distance (cm) | ns | ns |
| Axis Distance (cm/s) | ns | ns |
| Paw Drag (real#) | ns | ns |

### REFERENCES

1. Zu, T. et al. Non-ATG-initiated translation directed by microsatellite expansions. Proc Natl Acad Sci USA 108, 260-265, doi:10.1073/pnas.1013343108 (2011).
2. Ash, P. E. et al. Unconventional translation of C9ORF72 GGGGCC expansion generates insoluble polypeptides specific to c9FTD/ALS. Neuron 77, 639-646, doi:10.1016/j.neuron.2013.02.004 (2013).
3. Mori, K. et al. The C9orf72 GGGGCC repeat is translated into aggregating dipeptide-repeat proteins in FTLD/ALS. Science 339, 1335-1338, doi:10.1126/science.1232927 (2013).
4. Zu, T. et al. RAN proteins and RNA foci from antisense transcripts in C9ORF72 ALS and frontotemporal dementia. Proc Natl Acad Sci USA 110, E4968-4977, doi:10.1073/pnas.1315438110 (2013).
5. Cleary, J. D. & Ranum, L. P. New developments in RAN translation: insights from multiple diseases. Curr Opin Genet Dev 44, 125-134, doi:10.1016/j.gde.2017.03.006 (2017).
6. Green, K. M. et al. RAN translation at C9orf72-associated repeat expansions is selectively enhanced by the integrated stress response. Nat Commun 8, 2005, doi:10.1038/s41467-017-02200-0 (2017).
7. Cheng, W. et al. C9ORF72 GGGGCC repeat-associated non-AUG translation is upregulated by stress through eIF2alpha phosphorylation. Nat Commun 9, 51, doi:10.1038/s41467-017-02495-z (2018).
8. Todd, T. W. & Petrucelli, L. Insights into the pathogenic mechanisms of Chromosome 9 open reading frame 72 (C9orf72) repeat expansions. J Neurochem 138 Suppl 1, 145-162, doi:10.1111/jnc.13623 (2016).
9. Taylor, J. P., Brown, R. H. & Cleveland, D. W. Decoding ALS: from genes to mechanism. Nature 539, 197-206, doi:10.1038/nature20413 (2016).
10. Tian, B. et al. Expanded CUG repeat RNAs form hairpins that activate the double-stranded RNA-dependent protein kinase PKR. RNA 6, 79-87 (2000).
11. Sonenberg, N. & Hinnebusch, A. G. Regulation of translation initiation in eukaryotes: mechanisms and biological targets. Cell 136, 731-745, doi:10.1016/j.cell.2009.01.042 (2009).
12. Liu, Y. et al. C9orf72 BAC Mouse Model with Motor Deficits and Neurodegenerative Features of ALS/FTD. Neuron 90, 521-534, doi:10.1016/j.neuron.2016.04.005 (2016).
13. Benkirane, M. et al. Oncogenic potential of TAR RNA binding protein TRBP and its regulatory interaction with RNA-dependent protein kinase PKR. EMBO J 16, 611-624, doi:10.1093/emboj/16.3.611 (1997).
14. Park, H. et al. TAR RNA-binding protein is an inhibitor of the interferon-induced protein kinase PKR. Proc Natl Acad Sci USA 91, 4713-4717 (1994).
15. Pakos-Zebrucka, K. et al. The integrated stress response. EMBO Rep 17, 1374-1395, doi:10.15252/embr.201642195 (2016).
16. Barzilai, N., Crandall, J. P., Kritchevsky, S. B. & Espeland, M. A. Metformin as a Tool to Target Aging. Cell Metab 23, 1060-1065, doi:10.1016/j.cmet.2016.05.011 (2016).
17. Gantois, I. et al. Metformin ameliorates core deficits in a mouse model of fragile X syndrome. Nat Med 23, 674-677, doi:10.1038/nm.4335 (2017).
18. Ma, T. C. et al. Metformin therapy in a transgenic mouse model of Huntington's disease. Neurosci Lett 411, 98-103, doi:10.1016/j.neulet.2006.10.039 (2007).
19. Bañez-Coronel, M. et al. RAN Translation in Huntington Disease. Neuron 88, 667-677, doi:10.1016/j.neuron.2015.10.038 (2015).
20. Chen, Y. et al. Antidiabetic drug metformin (Glucophage®) increases biogenesis of Alzheimer's amyloid peptides via up-regulating BACE1 transcription. Proc Natl Acad Sci USA 106, 3907-3912, doi:10.1073/pnas.0807991106 (2009).
21. Foretz, M., Guigas, B., Bertrand, L., Pollak, M. & Viollet, B. Metformin: from mechanisms of action to therapies. Cell Metab 20, 953-966, doi:10.1016/j.cmet.2014.09.018 (2014).
22. Memmott, R. M. et al. Metformin prevents tobacco carcinogen-induced lung tumorigenesis. Cancer Prev Res (Phila) 3, 1066-1076, doi:10.1158/1940-6207.CAPR-10-0055 (2010).
23. Moon, S. L., Sonenberg, N. & Parker, R. Neuronal Regulation of eIF2alpha Function in Health and Neurological Disorders. Trends Mol Med 24, 575-589, doi:10.1016/j.molmed.2018.04.001 (2018).
24. Zhu, P. J. et al. Suppression of PKR Promotes Network Excitability and Enhanced Cognition by Interferon-gamma-Mediated Disinhibition. Cell 147, 1384-1396, doi:10.1016/j.cell.2011.11.029 (2011).
25. Kioumourtzoglou, M. A. et al. Diabetes Mellitus, Obesity, and Diagnosis of Amyotrophic Lateral Sclerosis: A Population-Based Study. JAMA Neurol 72, 905-911, doi:10.1001/jamaneurol.2015.0910 (2015).
26. Jawaid, A. et al. ALS disease onset may occur later in patients with pre-morbid diabetes mellitus. Eur J Neurol 17, 733-739, doi:10.1111/j.1468-1331.2009.02923.x (2010).
27. Zu, T. et al. RAN Translation Regulated by Muscleblind Proteins in Myotonic Dystrophy Type 2. Neuron 95, 1292-1305 e1295, doi:10.1016/j.neuron.2017.08.039 (2017).
28. 28. Ash P E, Bieniek K F, Gendron T F, Caulfield T, Lin W L, Dejesus-Hernandez M, van Blitterswijk M M, Jansen-West K, Paul J W, 3rd, Rademakers R et al. 2013. Unconventional translation of C9ORF72 GGGGCC expansion generates insoluble polypeptides specific to c9FTD/ALS. Neuron 77: 639-646.
29. 29. Banez-Coronel M, Ayhan F, Tarabochia A D, Zu T, Perez B A, Tusi S K, Pletnikova O, Borchelt D R, Ross C A, Margolis R L et al. 2015. RAN Translation in Huntington Disease. Neuron 88: 667-677.
30. Cleary J D, Ranum L P. 2017. New developments in RAN translation: insights from multiple diseases. Curr Opin Genet Dev 44: 125-134.
31. Mori K, Weng S M, Arzberger T, May S, Rentzsch K, Kremmer E, Schmid B, Kretzschmar H A, Cruts M, Van Broeckhoven C et al. 2013. The C9orf72 GGGGCC repeat is translated into aggregating dipeptide-repeat proteins in FTLD/ALS. Science 339: 1335-1338.
32. Todd P K, Oh S Y, Krans A, He F, Sellier C, Frazer M, Renoux A J, Chen K C, Scaglione K M, Basrur V et al. 2013. CGG repeat-associated translation mediates neurodegeneration in fragile X tremor ataxia syndrome. Neuron 78: 440-455.
33. Zu T, Gibbens B, Doty N S, Gomes-Pereira M, Huguet A, Stone M D, Margolis J, Peterson M, Markowski T W, Ingram M A et al. 2011. Non-ATG-initiated translation directed by microsatellite expansions. Proc Natl Acad Sci USA 108: 260-265.
34. Zu T, Liu Y, Banez-Coronel M, Reid T, Pletnikova O, Lewis J, Miller T M, Harms M B, Falchook A E, Subramony S H et al. 2013. RAN proteins and RNA foci from antisense transcripts in C9ORF72 ALS and frontotemporal dementia. Proc Natl Acad Sci USA 110: E4968-4977.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method of treating or preventing a neurological disease associated with repeat expansions in a subject in need thereof, the method comprising administering to the subject:
(a) a compound of Formula (I): 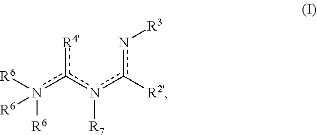
or a pharmaceutically acceptable salt, co-crystal, tautomer, stereoisomer, solvate, hydrate, polymorph, isotopically enriched derivative, or prodrug thereof, wherein:
each instance of [symbol] is a single bond or double bond, as valency permits;
R^{2′} is hydrogen, halogen, or —N(R^{2A})₂;
each instance of R^{2A} is independently hydrogen, optionally substituted acyl, optionally substituted alkyl, or a nitrogen protecting group;
R³ is hydrogen, optionally substituted alkyl, or a nitrogen protecting group;
R^{4′} is hydrogen, —N(R⁴)₂, or 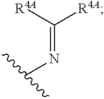
each instance of R⁴ is independently hydrogen, optionally substituted alkyl, a nitrogen protecting group, or absent, as valency permits; or optionally, when R^{4′} is —N(R⁴)₂, one instance of R⁴ is taken together with R³ and the intervening atoms to form an optionally substituted 5 to 7-membered heterocyclic ring;
each instance of R^{4A} is independently hydrogen, optionally substituted alkyl, or a nitrogen protecting group;
each instance of R⁶ is independently hydrogen, optionally substituted alkyl, a nitrogen protecting group, or absent, as valency permits; and
R⁷ is hydrogen, optionally substituted alkyl, a nitrogen protecting group, or absent, as valency permits; and
(b) either (i) a second compound of Formula (I) or (ii) compound of Formula (II): 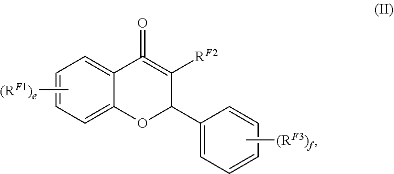
or a pharmaceutically acceptable salt, co-crystal, tautomer, stereoisomer, solvate, hydrate, polymorph, isotopically enriched derivative, or prodrug thereof, wherein:
each instance of R^{F1} is independently hydrogen, hydroxy, or alkoxy;
R^{F2} is hydrogen, hydroxy, or alkoxy;
each instance of R^{F3} is independently hydrogen, hydroxy, or alkoxy;
e is an integer selected from the group consisting of 0, 1, 2, 3, and 4; and
f is an integer selected from the group consisting of 0, 1, 2, 3, 4, and 5.

2. A method of treating or preventing a neurological disease associated with repeat expansions in a subject in need thereof, the method comprising administering to the subject a compound of Formula (II): 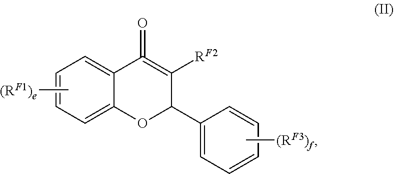
or a pharmaceutically acceptable salt, co-crystal, tautomer, stereoisomer, solvate, hydrate, polymorph, isotopically enriched derivative, or prodrug thereof, wherein:
each instance of R^{F1} is independently hydrogen, hydroxy, or alkoxy;
R^{F2} is hydrogen, hydroxy, or alkoxy;
each instance of R^{F3} is independently hydrogen, hydroxy, or alkoxy;
e is an integer selected from the group consisting of 0, 1, 2, 3, and 4; and
f is an integer selected from the group consisting of 0, 1, 2, 3, 4, and 5.

3. A pharmaceutical composition comprising:
(a) a compound of Formula (I): 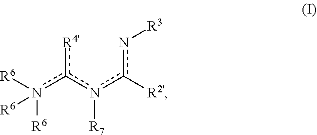
or a pharmaceutically acceptable salt, co-crystal, tautomer, stereoisomer, solvate, hydrate, polymorph, isotopically enriched derivative, or prodrug thereof, wherein:
each instance of [symbol] is a single bond or double bond, as valency permits;
R^{2′} is hydrogen, halogen, or —N(R^{2A})₂;
each instance of R^{2A} is independently hydrogen, optionally substituted acyl, optionally substituted alkyl, or a nitrogen protecting group;
R³ is hydrogen, optionally substituted alkyl, or a nitrogen protecting group;
R^{4′} is hydrogen, —N(R⁴)₂, or 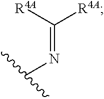
each instance of R⁴ is independently hydrogen, optionally substituted alkyl, a nitrogen protecting group, or absent, as valency permits; or optionally, when R^{4′} is —N(R⁴)₂, one instance of R⁴ is taken together with R³ and the intervening atoms to form an optionally substituted 5 to 7-membered heterocyclic ring;
each instance of R^{4A} is independently hydrogen, optionally substituted alkyl, or a nitrogen protecting group;
each instance of R⁶ is independently hydrogen, optionally substituted alkyl, a nitrogen protecting group, or absent, as valency permits; and
R⁷ is hydrogen, optionally substituted alkyl, a nitrogen protecting group, or absent, as valency permits; and
(b) either (i) a second compound of Formula (I) or (ii) compound of Formula (II): 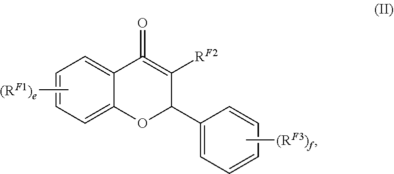
or a pharmaceutically acceptable salt, co-crystal, tautomer, stereoisomer, solvate, hydrate, polymorph, isotopically enriched derivative, or prodrug thereof, wherein:
each instance of R^{F1} is independently hydrogen, hydroxy, or alkoxy;
R^{F2} is hydrogen, hydroxy, or alkoxy;
each instance of R^{F3} is independently hydrogen, hydroxy, or alkoxy;
e is an integer selected from the group consisting of 0, 1, 2, 3, and 4; and
f is an integer selected from the group consisting of 0, 1, 2, 3, 4, and 5.

4. The method or composition of any one of claims 1-3, wherein:
each instance of R^{F1} is independently hydrogen, hydroxy, or methoxy;
R^{F2} is hydrogen or hydroxy; and
each instance of R^{F3} is independently hydrogen, hydroxy, or methoxy.

5. The method or composition of any one of claims 1-4, wherein each instance of R^{F1} is hydroxy.

6. The method or composition of any one of claims 1-5, wherein e is 2.

7. The method or composition of any one of claims 1-6, wherein each instance of R^{F3} is hydroxy.

8. The method or composition of any one of claims 1-7, wherein f is 2.

9. The method or composition of any one of claims 1-8, wherein the compound of Formula (II) is of formula: 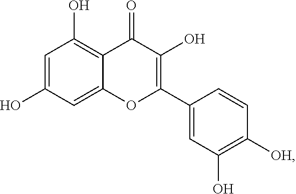 [SMILES: O=c1c(O)c(-c2ccc(O)c(O)c2)oc2cc(O)cc(O)c12]
or a pharmaceutically acceptable salt, co-crystal, tautomer, stereoisomer, solvate, hydrate, polymorph, isotopically enriched derivative, or prodrug thereof.

10. The method or composition of any one of claims 1-9, wherein the compound of Formula (II) is of formula: 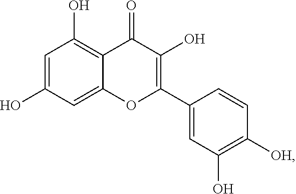 [SMILES: O=c1c(O)c(-c2ccc(O)c(O)c2)oc2cc(O)cc(O)c12]
or a pharmaceutically acceptable salt, co-crystal, tautomer, stereoisomer, solvate, hydrate, polymorph, isotopically enriched derivative, or prodrug thereof.

11. The method or composition of any one of claims 1 and 3-10, wherein R^{2′} is halogen.

12. The method or composition of any one of claims 1 and 3-11, wherein R^{2′} is I.

13. The method or composition of any one of claims 1 and 3-10, wherein R^{2′} is —N(R^{2A})₂.

14. The method or composition of claim 13, wherein each instance of R^{2A} is independently hydrogen or optionally substituted alkyl.

15. The method or composition of any one of claims 1 and 3-14, wherein R^{4′} is hydrogen.

16. The method or composition of any one of claims 1 and 3-14, wherein R^{4′} is

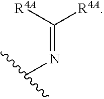

17. The method or composition of claim 16, wherein each instance of R^{4A} is independently hydrogen or optionally substituted alkyl.

18. The method or composition of any one of claims 1, 3-10 and 13-17, wherein the compound of Formula (I) or the second compound of Formula (I) is of formula: 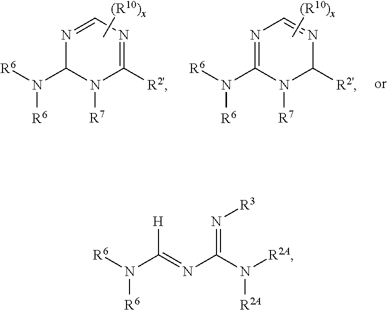
or a pharmaceutically acceptable salt, co-crystal, tautomer, stereoisomer, solvate, hydrate, polymorph, isotopically enriched derivative, or prodrug thereof, wherein:
x is 0 or 1; and
each instance of R¹⁰ is independently halogen, optionally substituted C₁₋₆ alkyl, —NH₂, —NH(optionally substituted C₁₋₆ alkyl), or —N(optionally substituted C₁₋₆ alkyl)₂.

19. The method or composition of claim any one of claims 1 and 3-18, wherein the compound of Formula (I) or the second compound of Formula (I) is of formula: 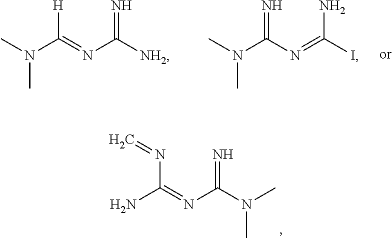
or a pharmaceutically acceptable salt, co-crystal, tautomer, stereoisomer, solvate, hydrate, polymorph, isotopically enriched derivative, or prodrug thereof.

20. The method or composition of any one of claims 1, 3-10, and 13, wherein the compound of Formula (I) or the second compound of Formula (I) is a compound of Formula (I-A): 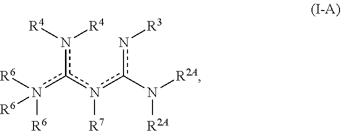
or a pharmaceutically acceptable salt, co-crystal, tautomer, stereoisomer, solvate, hydrate, polymorph, isotopically enriched derivative, or prodrug thereof, wherein:
each instance of [symbol] is a single bond or double bond, as valency permits;
each instance of R^{2A} is independently hydrogen, optionally substituted acyl, optionally substituted alkyl, or a nitrogen protecting group;
R³ is hydrogen, optionally substituted alkyl, or a nitrogen protecting group;
each instance of R⁴ is independently hydrogen, optionally substituted alkyl, a nitrogen protecting group, or absent, as valency permits; or, optionally, one instance of R⁴ is taken together with R³ and the intervening atoms to form an optionally substituted 5 to 7-membered heterocyclic ring;
each instance of R⁶ is independently hydrogen, optionally substituted alkyl, a nitrogen protecting group, or absent, as valency permits; and
R⁷ is hydrogen, optionally substituted alkyl, a nitrogen protecting group, or absent, as valency permits.

21. The method or composition of any one of claims 1, 3-10, 13, and 20, wherein the compound of Formula (I) or the second compound of Formula (I) is of formula: 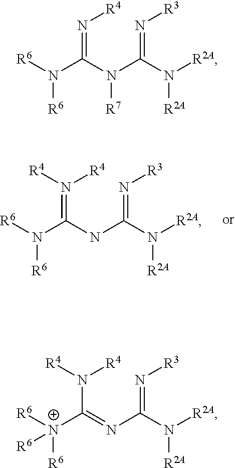
or a pharmaceutically acceptable salt, co-crystal, tautomer, stereoisomer, solvate, hydrate, polymorph, isotopically enriched derivative, or prodrug thereof.

22. The method or composition of any one of claims 1, 3-10, 13, and 20, wherein the compound of Formula (I) or the second compound of Formula (I) is of formula: 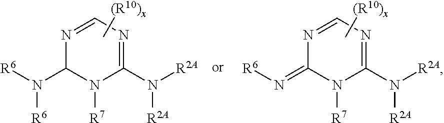
or a pharmaceutically acceptable salt, co-crystal, tautomer, stereoisomer, solvate, hydrate, polymorph, isotopically enriched derivative, or prodrug thereof, wherein:
x is 0 or 1; and
each instance of R¹⁰ is independently halogen, optionally substituted C₁₋₆ alkyl, —NH₂, —NH(optionally substituted C₁₋₆ alkyl), or —N(optionally substituted C₁₋₆ alkyl)₂.

23. The method or composition of claim 22, wherein x is 0.

24. The method or composition of claim 22, wherein x is 1.

25. The method or composition of claim 22 or 24, wherein at least one instance of R¹⁰ is optionally substituted C₁₋₆ alkyl.

26. The method or composition of any one of claims 1, 3-10, 13, and 20-21, wherein the compound of Formula (I) or the second compound of Formula (I) is of Formula (I-B): 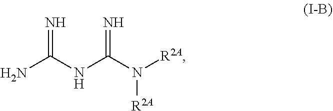
or a pharmaceutically acceptable salt, co-crystal, tautomer, stereoisomer, solvate, hydrate, polymorph, isotopically enriched derivative, or prodrug thereof, wherein:
each instance of R^{2A} is independently hydrogen, optionally substituted alkyl, or a nitrogen protecting group.

27. The method or composition of any one of claims 1, 3-10, and 13-26, wherein at least one instance of R^{2A} is hydrogen.

28. The method or composition of any one of claims 1, 3-10, and 13-26, wherein at least one instance of R^{2A} is optionally substituted C₁₋₆ alkyl.

29. The method or composition of any one of claims 1, 3-10, and 13-26, wherein at least one instance of R^{2A} is unsubstituted methyl or unsubstituted ethyl.

30. The method or composition of any one of claims 1, 3-21, and 26-29, wherein R³ is hydrogen.

31. The method or composition of any one of claims 1, 3-18, 20, 21, and 27-28, wherein R³ is optionally substituted C₁₋₆ alkyl.

32. The method or composition of any one of claims 1 and 3-31, wherein at least one instance of R⁶ is hydrogen.

33. The method or composition of any one of claims 1, 3-25, and 27-31, wherein at least one instance of R⁶ is optionally substituted C₁₋₆ alkyl.

34. The method or composition of any one of claims 1, 3-25, 27-31, and 33, wherein at least one instance of R⁶ is unsubstituted methyl.

35. The method or composition of any one of claims 1, 3-25, and 27-34, wherein R⁷ is absent.

36. The method or composition of any one of claims 1, 3-18, and 20-34, wherein R⁷ is hydrogen.

37. The method or composition of any one of claims 1-18, 20-25, and 27-34, wherein R⁷ is optionally substituted C₁₋₆ alkyl.

38. The method of claim 1 or composition of claim 3, wherein the compound of Formula (I) or the second compound of Formula (I) is of formula: 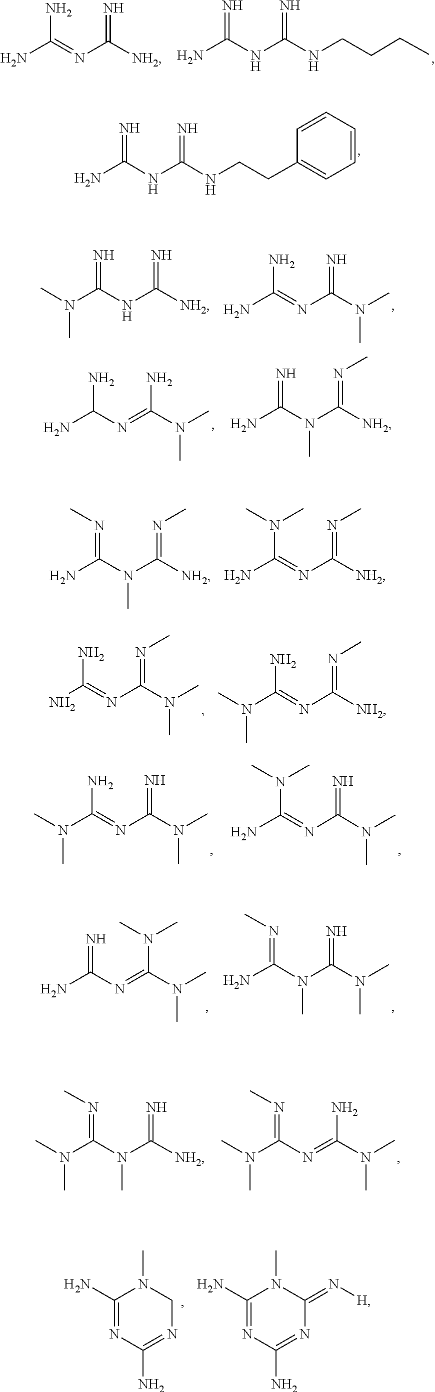 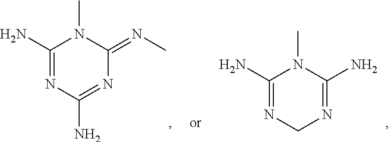
or a pharmaceutically acceptable salt, co-crystal, tautomer, stereoisomer, solvate, hydrate, polymorph, isotopically enriched derivative, or prodrug thereof.

39. The method of claim 1 or composition of claim 3, wherein:
(a) the compound of Formula (I) is: 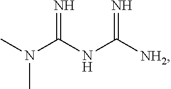
or a pharmaceutically acceptable salt, co-crystal, tautomer, stereoisomer, solvate, hydrate, polymorph, isotopically enriched derivative, or prodrug thereof; and
(b) the compound of Formula (II) is: 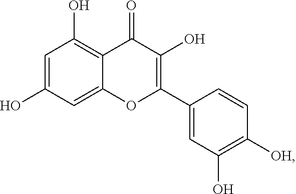 [SMILES: O=c1c(O)c(-c2ccc(O)c(O)c2)oc2cc(O)cc(O)c12]
or a pharmaceutically acceptable salt, co-crystal, tautomer, stereoisomer, solvate, hydrate, polymorph, isotopically enriched derivative, or prodrug thereof.

40. The method of claim 1 or composition of claim 3, wherein:
(a) the compound of Formula (I) is 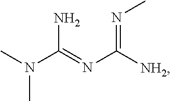
or a pharmaceutically acceptable salt, co-crystal, tautomer, stereoisomer, solvate, hydrate, polymorph, isotopically enriched derivative, or prodrug thereof; and
(b) the compound of Formula (II) is 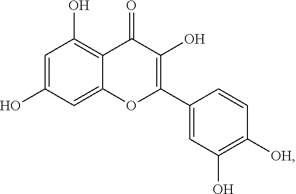 [SMILES: O=c1c(O)c(-c2ccc(O)c(O)c2)oc2cc(O)cc(O)c12]
or a pharmaceutically acceptable salt, co-crystal, tautomer, stereoisomer, solvate, hydrate, polymorph, isotopically enriched derivative, or prodrug thereof.

41. The method of claim 1 or composition of claim 3, wherein:
(a) the compound of Formula (I) is 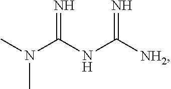
or a pharmaceutically acceptable salt, co-crystal, tautomer, stereoisomer, solvate, hydrate, polymorph, isotopically enriched derivative, or prodrug thereof; and
(b) the compound of Formula (II) is 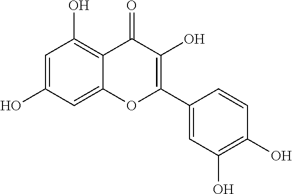 [SMILES: O=c1c(O)c(-c2ccc(O)c(O)c2)oc2cc(O)cc(O)c12]
or a pharmaceutically acceptable salt, co-crystal, tautomer, stereoisomer, solvate, hydrate, polymorph, isotopically enriched derivative, or prodrug thereof.

42. The method of claim 1 or composition of claim 3, wherein:
(a) the compound of Formula (I) is 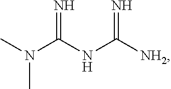
or a pharmaceutically acceptable salt, co-crystal, tautomer, stereoisomer, solvate, hydrate, polymorph, isotopically enriched derivative, or prodrug thereof; and
(b) the second compound of Formula (I) is 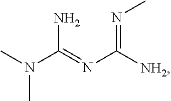
or a pharmaceutically acceptable salt, co-crystal, tautomer, stereoisomer, solvate, hydrate, polymorph, isotopically enriched derivative, or prodrug thereof.

43. The method or composition of any one of claims 1-42, wherein the compound of Formula (I) and/or the compound of Formula (II) or the second compound of Formula (I) is in the form of a compound, or pharmaceutically acceptable salt, tautomer, solvate, hydrate, isotopically enriched derivative, or prodrug thereof.

44. The method or composition of any one of claims 1-43, wherein the compound of Formula (I) and/or the compound of Formula (II) or the second compound of Formula (I) is in the form of a compound, or pharmaceutically acceptable salt or tautomer thereof.

45. The method of any one of claims 1 and 4-44, wherein the method comprises administering to the subject a dose of 200-1000 mg per day of the compound of Formula (I), or a pharmaceutically acceptable salt, co-crystal, tautomer, stereoisomer, solvate, hydrate, polymorph, isotopically enriched derivative, or prodrug thereof, and/or the second compound of Formula (I), or a pharmaceutically acceptable salt, co-crystal, tautomer, stereoisomer, solvate, hydrate, polymorph, isotopically enriched derivative, or prodrug thereof.

46. The method of any one of claims 1, 2, and 4-45, wherein the compound is administered over a period of at least 10 days, at least 30 days, at least six months, or at least one year.

47. The method of any one of claims 1, 2, and 4-46, wherein the method treats a neurological disease in a subject.

48. The method of any one of claims 1, 2, and 4-46, wherein the method prevents a neurological disease in a subject.

49. The method of any one of claims 1, 2, and 4-48, whereby the method reduces the level of one or more repeat associated non-AUG (RAN) proteins in the subject.

50. The method of any one of claims 1, 2, and 4-49, whereby the method reduces the accumulation of one or more repeat associated non-AUG (RAN) proteins in the subject.

51. The method of any one of claims 1, 2, and 4-50, wherein the method reduces the translation of one or more repeat associated non-AUG (RAN) proteins.

52. The method of any one of claims 1, 2, and 4-51, wherein the method inhibits the translation of one or more repeat associated non-AUG (RAN) proteins.

53. The method of any one of claims 1, 2, and 4-52, wherein the one or more RAN proteins are selected from the group consisting of poly(Alanine) [polyAla or poly(A)]; poly(Arginine) [polyArg or poly(R)]; poly(Arginine-Glutamate) [poly(RE)]; poly(Cysteine) [polyCys or poly(C)]; poly(Cysteine-Proline) [poly(CP)]; poly(Glutamine) [polyGln or poly(Q)]; poly(Glutamine-Alanine) [poly(QA)]; poly(Glycine) [poly(G)]; poly(Glycine-Alanine) [poly(GA)]; poly(Glycine-Arginine) [poly(GR)]; poly(Glycine-Aspartate) [poly(GD)]; poly(Glycine-Glutamate) [poly(GE)]; poly(Glycine-Glutamine) [poly(GQ)]; poly(Glycine-Leucine) [poly(GL)]; poly(Glycine-Lysine) [poly(GK)]; poly(Glycine-Proline) [(poly(GP)]; poly(Glycine-Threonine) [poly(GT)]; poly(Leucine) [polyLeu or poly(L)]; poly(Leucine-Proline) [poly(LP)]; poly(Leucine-Serine) [poly(LS)]; poly(phenylalanine-proline) [poly(FP)]; poly(Proline) [poly(P)]; poly(Proline-Alanine) [poly(PA)]; poly(Proline-Arginine) [poly(PR)]; poly(Serine) [polySer]; poly(Serine-Proline) [poly(SP)]; poly(Tryptophan-Alanine) [poly(WA)]; poly(Valine-Proline) [poly(VP)]; poly(Leucine-Proline-Alanine-Cysteine) [poly(LPAC)]; poly(Glutamine-Alanine-Glycine-Arginine) [poly(QAGR)]; Poly(Isoleucine-Leucine-Phenylalanine-Tyrosine-Serine) [poly(ILFTS)]; Poly(Phenylalanine-Histidine-Serine-Isoleucine-Proline) [poly(FHSIP)]; Poly(Tryptophan-Asparagine-Glycine-Methionine-Glutamine) [poly(WNGMQ)]; poly(PGGRGE); poly(FTPLSLPV); poly(LLPSPSRC); poly(YSPLPPGV); poly(HREGEGSK); poly(TGRERGVN); poly(GRQRGVNT); and poly(GSKHREAE), or combinations thereof.

54. The method of any one of claims 1, 2, and 4-53, wherein the one or more RAN proteins comprises at least 35 poly-amino acid repeats in the RAN protein.

55. The method of any one of claims 1, 2, and 4-54, wherein the repeat expansions comprise AGGAT expansions, ATCCT expansions, ATCCT expansions, CAG expansions, CAGG expansions, CCAAGA expansions, CCAAGG expansions, CCACGA expansions, CCACGC expansions, CCACGG expansions, CCACGT expansions, CCCAGA expansions, CCCAGG expansions, CCCCGA expansions, CCCCGC expansions, CCCCGG expansions, CCCCGT expansions, CCGAGA expansions, CCGAGG expansions, CCGCGA expansions, CCGCGC expansions, CCGCGG expansions, CCGCGT expansions, CCTAGA expansions, CCTAGG expansions, CCTCGA expansions, CCTCGC expansions, CCTCGG expansions, CCTCGT expansions, CCTG expansions, CTG expansions, GAGAGG expansions, GGCCCA expansions, GGCCCC expansions, GGCCTG expansions, GGGGCA expansions, GGGGCC expansions, TGGAA expansions, and/or TGGGCC expansions.

56. The method of any one of claims 1, 2, and 4-54, wherein the repeat expansions comprise CAG expansions and CTG expansions.

57. The method of any one of claims 1, 2, and 4-54, wherein the repeat expansions comprise CAGG expansions and CCTG expansions.

58. The method of any one of claims 1, 2, and 4-54, wherein the repeat expansions comprise TGGAA expansions or GGGGCC expansions and GGCCCC expansions.

59. The method of any one of claims 1, 2, and 4-54, wherein the repeat expansions comprise GGCCTG expansions, TGGGCC expansions, or GGCCCA expansions.

60. The method of any one of claims 1, 2, and 4-54, wherein the repeat expansions comprise ATCCT expansions, ATCCT expansions, or AGGAT expansions.

61. The method of any one of claims 1, 2, and 4-60, wherein the neurological disease is a neurodegenerative disorder.

62. The method of any one of claims 1, 2, and 4-61, wherein the neurological disease is Alzheimer's disease.

63. The method of any one of claims 1, 2, and 4-61, wherein the neurological disease is amyotrophic lateral sclerosis (ALS) or frontotemporal dementia (FTD).

64. The method of claim 63, wherein the neurological disease is C9ORFf72 ALS or C9ORF72 FTD.

65. The method of any one of claims 1, 2, and 4-64, wherein the neurological disease is spinocerebellar ataxia.

66. The method of claim 65, wherein the spinocerebellar ataxia is spinocerebellar ataxia type 1, spinocerebellar ataxia type 2, spinocerebellar ataxia type 3, spinocerebellar ataxia type 8, spinocerebellar ataxia type 6, spinocerebellar ataxia type 7, spinocerebellar ataxia type 10, spinocerebellar ataxia type 12, spinocerebellar ataxia type 17, spinocerebellar ataxia type 31, or spinocerebellar ataxia type 36.

67. The method of any one of claims 1, 2, and 4-61, wherein the neurological disease is myotonic dystrophy type 1, myotonic dystrophy type 2, or Fuch's corneal endothelial dystrophy.

68. The method of any one of claims 1, 2, and 4-61, wherein the neurological disease is spinal bulbar muscular atrophy or dentatorubral-pallidoluysian atrophy.

69. The method of any one of claims 1, 2, and 4-61, wherein the neurological disease is Huntington's disease.

70. The method of any one of claims 1, 2, and 4-61, wherein the neurological disease is Fragile X Tremor Ataxia Syndrome (FXTAS).

71. The method of any one of claims 1, 2, 4-59 and 64, wherein the neurological disease is Huntington's disease-like 2 syndrome (HDL2); Fragile X syndrome (FXS); disorders related to 7p11.2 folate-sensitive fragile site FRA7A; disorders related to folate-sensitive fragile site 2q11 FRA2A; or Fragile XE syndrome (FRAXE).

72. The method of any one of claims 1, 2, and 4-71 further comprising administering an additional therapeutic agent for treating a neurological disease.

73. The method of any one of claims 1 and 4-72, wherein (a) the compound of Formula (I) and (b) the second compound of Formula (I) are administered in separate pharmaceutical compositions.

74. The method of any one of claims 1 and 4-73, wherein (a) the compound of Formula (I) and (b) the second compound of Formula (I) are administered concurrently.

75. The method of any one of claims 1 and 4-73, wherein (a) the compound of Formula (I) and (b) the second compound of Formula (I) are administered sequentially.

76. The method of claim 75, wherein the compound of Formula (I) is administered before the second compound of Formula (I).

77. The method of claim 75, wherein the second compound of Formula (I) is administered before the compound of Formula (I).

78. The method of any one of claims 1 and 4-77, wherein the administration of the combination of compounds exhibits synergy compared to administration of the compound of Formula (I) or the second compound of Formula (I) alone.

79. The method of any one of claims 1 and 4-78, wherein a lower effective dose of the compound of Formula (I) and/or the second compound of Formula (I) may be administered as compared to when said compounds are administered alone.

80. The method or composition of any one of claims 1 and 3-78, wherein the ratio of the compound of Formula (I) to the second compound of Formula (I) is about 1:1, about 1:2, about 1:2.5, about 1:4, about 1:5, about 1:8, about 1:16, about 16:1, about 8:1, about 5:1, about 4:1, about 1:2.5, or about 2:1.

81. The method of any one of claims 1 and 4-72, wherein (a) the compound of Formula (I) and (b) the compound of Formula (II) are administered in separate pharmaceutical compositions.

82. The method of any one of claims 1, 4-72, and 81, wherein (a) the compound of Formula (I) and (b) the compound of Formula (II) are administered concurrently.

83. The method of any one of claims 1, 4-72, and 81, wherein (a) the compound of Formula (I) and (b) the compound of Formula (II) are administered sequentially.

84. The method of claim 83, wherein the compound of Formula (I) is administered before the compound of Formula (II).

85. The method of claim 83, wherein the compound of Formula (II) is administered before the compound of Formula (I).

86. The method of any one of claims 1, 4-72, and 81-85, wherein the administration of the combination of compounds exhibits synergy compared to administration of the compound of Formula (I) or the compound of Formula (II) alone.

87. The method of any one of claims 1, 4-72, and 81-86, wherein a lower effective dose of the compound of Formula (I) and/or the compound of Formula (II) may be administered as compared to when said compounds are administered alone.

88. The method or composition of any one of claims 1, 3-72 and 81-87, wherein the ratio of the compound of Formula (I) to or the compound of Formula (II) is about 1:1, about 1:2, about 1:2.5, about 1:4, about 1:5, about 1:8, about 1:16, about 16:1, about 8:1, about 5:1, about 4:1, about 1:2.5, or about 2:1.

89. The method or composition of claim 80 or 88, wherein the ratio is about 1:2, about 1:4, about 1:8, or about 1:16.

90. The method or composition of any one of claims 80, 88, and 89, wherein the ratio is about 1:18 or about 1:16.

91. The composition of any one of claims 3-42, 80, and 88-90 further comprising a pharmaceutically acceptable excipient.

92. The composition of any one of claims 3-42, 80, and 88-91 further comprising a third compound selected from the group consisting of a compound of Formula (I), or a pharmaceutically acceptable salt, co-crystal, tautomer, stereoisomer, solvate, hydrate, polymorph, isotopically enriched derivative, or prodrug thereof, and a compound of Formula (II), or a pharmaceutically acceptable salt, co-crystal, tautomer, stereoisomer, solvate, hydrate, polymorph, isotopically enriched derivative, or prodrug thereof.

93. A kit for treating a neurological disease associated with repeat expansions in a subject in need thereof comprising:
a compound of Formula (I), or a pharmaceutically acceptable salt, co-crystal, tautomer, stereoisomer, solvate, hydrate, polymorph, isotopically enriched derivative, or prodrug thereof;
a compound of Formula (II), or a pharmaceutically acceptable salt, co-crystal, tautomer, stereoisomer, solvate, hydrate, polymorph, isotopically enriched derivative, or prodrug thereof; and
instructions for administering the compound of Formula (I), or the pharmaceutically acceptable salt, co-crystal, tautomer, stereoisomer, solvate, hydrate, polymorph, isotopically enriched derivative, or prodrug thereof, and the compound of Formula (II), or the pharmaceutically acceptable salt, co-crystal, tautomer, stereoisomer, solvate, hydrate, polymorph, isotopically enriched derivative, or prodrug thereof.

94. A kit for treating a neurological disease associated with repeat expansions in a subject in need thereof comprising:
a compound of Formula (II), or a pharmaceutically acceptable salt, co-crystal, tautomer, stereoisomer, solvate, hydrate, polymorph, isotopically enriched derivative, or prodrug thereof; and
instructions for administering the compound of Formula (II), or the pharmaceutically acceptable salt, co-crystal, tautomer, stereoisomer, solvate, hydrate, polymorph, isotopically enriched derivative, or prodrug thereof.

95. A kit for treating a neurological disease associated with repeat expansions in a subject in need thereof comprising:
the composition of any one of claims 3-42, 80, and 88-92; and
instructions for administering the composition of any one of claims 3-42, 80, and 88-92.

96. Use of: (a) a compound of Formula (I): 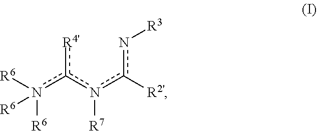
or a pharmaceutically acceptable salt, co-crystal, tautomer, stereoisomer, solvate, hydrate, polymorph, isotopically enriched derivative, or prodrug thereof, wherein:
each instance of [symbol] is a single bond or double bond, as valency permits;
R^{2′} is hydrogen, halogen, or —N(R^{2A})₂;
each instance of R^{2A} is independently hydrogen, optionally substituted acyl, optionally substituted alkyl, or a nitrogen protecting group;
R³ is hydrogen, optionally substituted alkyl, or a nitrogen protecting group;
R^{4′} is hydrogen, —N(R⁴)₂, or 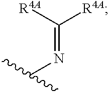
each instance of R⁴ is independently hydrogen, optionally substituted alkyl, a nitrogen protecting group, or absent, as valency permits; or optionally, when R^{4′} is —N(R⁴)₂, one instance of R⁴ is taken together with R³ and the intervening atoms to form an optionally substituted 5 to 7-membered heterocyclic ring;
each instance of R^{4A} is independently hydrogen, optionally substituted alkyl, or a nitrogen protecting group;
each instance of R⁶ is independently hydrogen, optionally substituted alkyl, a nitrogen protecting group, or absent, as valency permits; and
R⁷ is hydrogen, optionally substituted alkyl, a nitrogen protecting group, or absent, as valency permits; and
(b) either (i) a second compound of Formula (I) or (ii) compound of Formula (II): 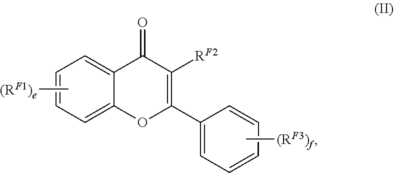
or a pharmaceutically acceptable salt, co-crystal, tautomer, stereoisomer, solvate, hydrate, polymorph, isotopically enriched derivative, or prodrug thereof, wherein:
each instance of R^{F1} is independently hydrogen, hydroxy, or alkoxy;
R^{F2} is hydrogen, hydroxy, or alkoxy;
each instance of R^{F3} is independently hydrogen, hydroxy, or alkoxy;
e is an integer selected from the group consisting of 0, 1, 2, 3, and 4; and
f is an integer selected from the group consisting of 0, 1, 2, 3, 4, and 5,
for treating or preventing a neurological disease associated with repeat expansions.

97. Use of a compound of Formula (II): 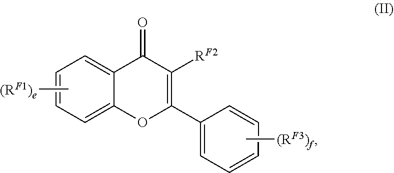
or a pharmaceutically acceptable salt, co-crystal, tautomer, stereoisomer, solvate, hydrate, polymorph, isotopically enriched derivative, or prodrug thereof, wherein:
each instance of R^{F1} is independently hydrogen, hydroxy, or alkoxy;
R^{F2} is hydrogen, hydroxy, or alkoxy;
each instance of R^{F3} is independently hydrogen, hydroxy, or alkoxy;
e is an integer selected from the group consisting of 0, 1, 2, 3, and 4; and
f is an integer selected from the group consisting of 0, 1, 2, 3, 4, and 5,
for treating or preventing a neurological disease associated with repeat expansions.
